(19) United States
(12) Patent Application Publication
(10) Pub. No.: US 2024/0010980 A1
(43) Pub. Date: Jan. 11, 2024

(54) NATURAL KILLER CELLS WITH ENHANCED ACTIVITY

(71) Applicant: The Regents of the University of California, Oakland, CA (US)

(72) Inventors: Dan S. Kaufman, La Jolla, CA (US); Kenta Yamamoto, La Jolla, CA (US)

(21) Appl. No.: 18/249,991

(22) PCT Filed: Oct. 27, 2021
(86) PCT No.: PCT/US2021/056761

§ 371 Date: Apr. 21, 2023

Related U.S. Application Data
(60) Provisional application No. 63/105,978, filed on Oct. 27, 2020.

(51) Int. Cl. C12N 5/0783; C12N 9/64; A61K 35/17; C07K 16/28; A61P 35/00; A61K 35/545

(52) U.S. Cl. CPC C12N 5/0646; C12N 9/6489; C12Y 304/24086; A61K 35/17; C07K 16/2887; C07K 16/2863; A61P 35/00; A61K 35/545; C12N 2506/45; C12N 2510/00; C07K 2317/732; A61K 2039/505

(57) ABSTRACT

Engineered immune cells, optionally natural killer (NK) cells, comprising an inactivating mutation for an endogenous A-Disintegrin-And-Metalloproteinase 17 (ADAM 17) gene. ADAM17-deficient immune cells produced from stem cells, optionally, induced pluripotent stem cells (iPSCs), having enhanced antibody-dependent cellular cytotoxicity (ADCC). Pharmaceutical compositions comprising said engineered immune cells. Methods for making the engineered immune cell and pharmaceutical compositions, and methods of use are provided.

### CROSS REFERENCE TO RELATED APPLICATIONS

This application claims the priority benefit of U.S. Provisional Application No. 63/105,978 filed Oct. 27, 2020, which application is incorporated herein by reference.

### GOVERNMENT SPONSORSHIP

This invention was made with government support under grant No. CA203348 awarded by the National Institutes of Health. The government has certain rights in the invention.

### TECHNICAL FIELD

The present invention relates generally to natural killer (NK) cells with enhanced activity. The NK cells may be derived from stem cells. Other aspects and advantages of the invention will be readily apparent from the following detailed description of the invention.

### BACKGROUND

Adoptive cell therapy is rapidly gaining interest as a promising new method to treat cancer. In particular, chimeric antigen receptor (CAR) T cells, the only U.S. F.D.A.-approved lymphocyte-based adoptive cancer cell therapy to treat cancer recently approved in 2017, have shown remarkable efficacy in treating refractory B cell malignancies. Success of CAR-T cell therapy has fueled optimism for the development of more effective adoptive cell therapy options. Currently approved CAR-T treatment regimens rely on autologous transplantation of ex vivo modified and expanded T cells harvested through leukapheresis from the original patients. This process takes 3-4 weeks, and donor variability on the quality of harvested T cells from each individual patient can widely affect treatment outcome. Furthermore, some patients receiving CAR-T cell therapy experience potentially lethal side effects, notably cytokine release syndrome (CRS) and neurotoxicity. Thus, development of standardized, “off-the-shelf” cell therapy products with defined, consistent quality that can be administered into patients in a timely manner with minimal side effects is highly desirable and is of great commercial interest.

In this context, focus is turning to natural killer (NK) cells as a suitable cell source for “off-the-shelf” cell therapy. Unlike T cells, NK cells possess a native ability to kill tumors and virally infected cells without prior antigen priming. Furthermore, NK cells can be administered to patients across HLA allotypes, unlike T cells which require HLA matching to avoid graft-versus-host disease. Many trials utilizing adoptive transfer of allogeneic NK cells demonstrated complete remissions in patients with acute myelogenous leukemia (AML) who are refractory to standard chemotherapy. Another recent clinical study demonstrated effective treatment of lymphoid malignancies using allogeneic CAR-expressing NK cells, with minimal side effects. Thus, NK cells possess a number of advantages over T cells that enables them to be used as safe, effective, “off-the-shelf” adoptive cell therapy product to treat diverse malignancies.

In addition to NK cells sourced from donor peripheral blood (PB), cord blood (CB), and established NK tumor cell lines, NK cells generated from induced pluripotent stem cells (iPSCs) is a promising source for allogeneic NK cells. Most notably, iPSCs are more amenable to genetic modifications—namely gene deletions/mutations or over-expression, which can be harnessed to enhance the cytotoxic activity of the NK cells to mediate improved anti-tumor activity. In contrast, CB and PB-derived NK cells are more difficult to genetically manipulate, produce a more heterogeneous gene-modified cell population, and donor variability can also affect downstream cell therapy efficacy. Human iPSCs also grow indefinitely in culture in an undifferentiated state, and methods pioneered and refined to derive NK cells from iPSCs now allow for the production of iPSC-derived NK cells at clinical scale.

Therefore, iPSCs serves as an ideal template for generating standardized, gene-edited, and uniform adoptive NK cell therapy with enhanced cytotoxicity. Accordingly, several high-profile biotech and pharmaceutical companies are actively developing “off-the-shelf” iPSC-derived NK cell therapy products, and some have already entered clinical trials. Identifying genetic modification strategies to enhance NK cell-mediated cytotoxicity is a very active area of investigation with tremendous commercial implications, as this allows for the generation of a more potent adoptive NK cell therapy product to treat a variety of malignancies.

As such, NK cells can be useful in adoptive cell therapies, however their use is often limited by biological constraints and results in suboptimal efficacy. Therefore, there is an unmet need for compositions comprising said cells and methods of their use.

### SUMMARY OF THE INVENTION

In an aspect, the disclosure provides an engineered immune cell, comprising an inactivating mutation in an endogenous A-Disintegrin-And-Metalloproteinase 17 (ADAM17) gene.

In some embodiments, the engineered immune cell is an induced pluripotent stem cell (iPSC)-derived immune cell. In some embodiments, the engineered immune cell is a peripheral blood (PB)-derived immune cell. In some embodiments, the engineered immune cell is a cord blood (CB)-derived immune cell.

In some embodiments, the engineered immune cell is a natural killer (NK) cell.

In some embodiments, the ADAM17 inactivating mutation is a knockout of an endogenous ADAM17 gene. In some embodiments, the engineered immune cell is a ADAM17^{-/-} immune cell. In some embodiments, the ADAM17 inactivating mutation is a knockdown of an endogenous ADAM17 gene.

In some embodiments, the engineered immune cell is CD56+, CD94+, NKG2D+, NKp44+, and NKp46+.

In some embodiments, the engineered immune cell has increased expression of CD16a, TNF-α, and CD62L (L-selectin) as compared to a non-engineered immune cell after stimulation with a stimulating agent.

In some embodiments, expression of CD16a in the engineered immune cell is stably maintained for a period of at least about 6 weeks during expansion in a culture.

In some embodiments, the immune cell is a human immune cell.

In an aspect, the disclosure provides a purified cell composition comprising one or more of the engineered immune cell of the disclosure.

In some embodiments, at least about 71% of engineered immune cells express tumor necrosis factor-alpha (TNF-α) after stimulation with a stimulating agent. In some embodiments, at least about 42% of engineered immune cells express CD62L (L-selectin) after stimulation with a stimulating agent.

In some embodiments, the engineered immune cells have increased expression of CD107a and interferon gamma (IFNγ) as compared to non-engineered immune cells after co-incubation with a disease cell line and an antibody specific to said disease cell line.

In some embodiments, disease cell line is a B-lymphoma cell line and the antibody specific to said disease cell line is an anti-CD20 antibody. In some embodiments, the anti-CD20 antibody is rituximab. In some embodiments, the engineered immune cells exhibit at least about 45% increased expression of CD107a. In some embodiments, the engineered immune cells exhibit at least about 36% increased expression of IFNγ.

In some embodiments, the disease cell line is a squamous cell carcinoma cell line and the antibody specific to said disease cell line is an anti-EGFR antibody. In some embodiments, the anti-EGFR antibody is cetuximab. In some embodiments, the engineered immune cells exhibit at least about 29% increased expression of CD107a. In some embodiments, the engineered immune cells exhibit at least about 39% increased expression of IFNγ.

In some embodiments, the engineered immune cells exhibit enhanced antibody-dependent cellular cytotoxicity (ADCC) as compared to non-engineered immune cells.

In an aspect, the disclosure provides a method of making the engineered immune cell of the disclosure comprising: a) introducing an inactivating mutation in an endogenous A-Disintegrin-And Metalloproteinase 17 (ADAM17) gene into a stem cell; and b) differentiating the stem cell into an immune cell. In some embodiments, the stem cell is an induced pluripotent stem cell (iPSC), peripheral blood cell, or a cord blood cell. In some embodiments, the immune cell is a natural killer cell (NK) cell.

In an aspect, the disclosure provides a pharmaceutical composition comprising the engineered immune cell of the disclosure and one or more pharmaceutically acceptable excipients or diluents.

In an aspect, the disclosure provides a kit comprising the engineered immune cell of the disclosure or the pharmaceutical composition of the disclosure and instructions for use.

In an aspect, the disclosure provides a method of treating or preventing a disease or disorder in a subject in need thereof, comprising administering the engineered immune cell of the disclosure or the pharmaceutical composition of the disclosure to the subject. In some embodiments, the disease or disorder is a malignancy. In some embodiments, the malignancy comprises a tumor-associated antigen. In some embodiments, the disease or disorder is a viral infection. In some embodiments, the viral infection comprises a viral infection-associated antigen.

In some embodiments, the administering further comprises administering the engineered immune cell or pharmaceutical composition comprising said engineered immune cell in combination with an antibody specific to a disease. In some embodiments, the antibody specific to a disease is an anti-CD20 antibody. In some embodiments, the anti-CD20 antibody is rituximab. In some embodiments, the antibody specific to a disease is an anti-EGFR antibody. In some embodiments, the anti-EGFR antibody is cetuximab.

In an aspect, the disclosure provides a cellular culture comprising genetically modified ADAM17-deficient Natural Killer (NK) cells. In embodiments, the NK cells have been produced from induced pluripotent stem cells (iPSCs). In embodiments, the NK cells have been produced from peripheral blood cells or cord blood cells. In embodiments, the NK cells are human NK cells.

In some embodiments, the NK cells exhibit enhanced antibody-dependent cellular cytotoxicity (ADCC) as compared to non-genetically modified NK cells.

In embodiments, the NK cells express cell surface markers CD56, NKG2D, NKp44, and NKp46. In embodiments, the invention provides that the NK cells express increased cell surface markers CD16, TNF-α, and CD62L as compared to non-genetically modified NK cells.

In embodiments, the NK cells have been genetically modified to be ADAM17-deficient with CRISPR, TALEN, ZFN or other gene editing techniques.

In an aspect, the disclosure provides a pharmaceutical composition comprising NK cells from the culture of genetically modified ADAM17-deficient NK cells as described herein.

In an aspect, the disclosure provides a method of treating a subject in need comprising administering to the subject an effective amount of a pharmaceutical composition as described herein. In embodiments, the invention provides that the subject in need has a NK-resistant cancer. In embodiments, the invention provides that the subject in need has a chronic viral infection.

In embodiments, the administration further includes antibodies specific for a diseased cell. In embodiments, the invention provides that the administration further includes antibodies specific for CD20.

In an aspect, the disclosure provides a method of manufacturing a NK cell culture as described herein comprising genetically modifying a cell to be ADAM17-deficient. In some embodiments, the cell is an induced pluripotent stem cell (iPSC).

### BRIEF DESCRIPTION OF THE DRAWINGS

FIG. 1 shows a schematic of an embodiment of the present invention.

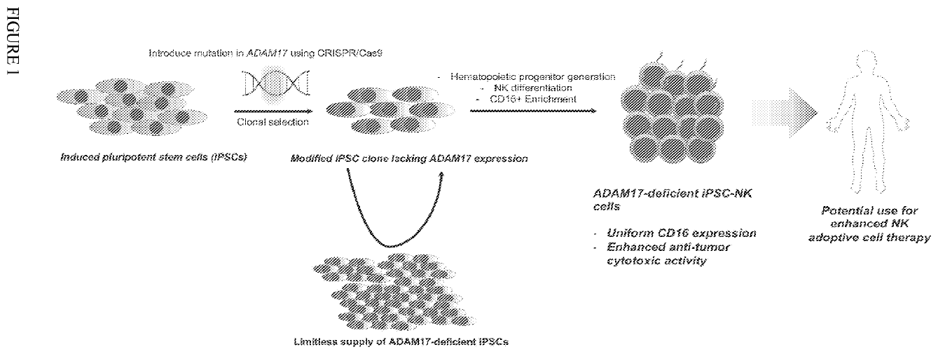

FIG. 2 shows that ADAM17-KO iPSC-NK cells demonstrate enhanced cytotoxicity against RA JI cells in the presence of rituximab (anti-CD20).

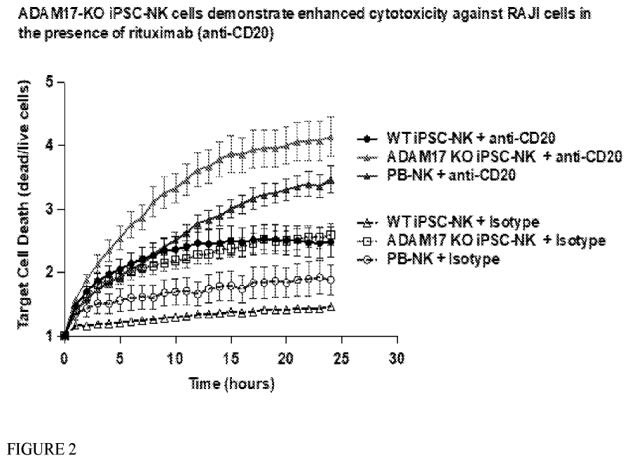

FIGS. 3A-3F show ADAM17-deficient iPSCs efficiently differentiate to hematopoietic progenitor cells and functional NK cells. FIG. 3A shows genomic location for targeting exon 1 in ADAM17 using CRISPR/Cas9. Sequence in red denotes the non-limiting exemplary short guide RNA (sgRNA) sequence used, and corresponding encoded amino acids within the region below. Numeric genomic location on chromosome 2 corresponding to the sgRNA sequence based on human genome assembly GRCh38.p13. FIG. 3B shows surface marker expression for pluripotency markers S SEA-4 and TRA-1-81, and ADAM17 on wildtype control (WT) and ADAM17-deficient (ADAM17-KO) iPSCs. Red greyscales histogram—isotype control. Blue greyscales histogram—stain for indicated markers. FIG. 3C shows surface marker expression for CD34, CD31, CD43, CD45 on dissociated spin embryoid bodies (EB) generated from WT and ADAM17 KO iPSCs 7 days post-seeding. FIG. 3D shows representative surface marker expression of iPSC-NK cells generated from WT and ADAM17-KO iPSCs, and healthy donor peripheral blood NK cells (PB-NK). Red greyscales histogram—isotype control. Blue greyscales histogram—stain for indicated markers. FIG. 3E shows quantification of % CD16⁺ cells (as in (C), CD16 panel) from multiple experiments analyzing CD16 surface expression on WT iPSC-NKs and ADAM17 KO iPSC-NKs generated after EB formation and 27-35 days in NK differentiation conditions. Each dot represents a biological replicate of iPSC-NK cell batch generated. **P<0.005 as determined by Student's two-tailed t-test assuming equal variances. Error bars indicate ±S.D. FIG. 3F shows 4-hour cytotoxicity assay measuring cell death and apoptosis (Caspase 3/7, 7-AAD) after co-incubating K562 erythroleukemia cells and indicated NK cells at indicated effector-to-target (E:T) ratios.

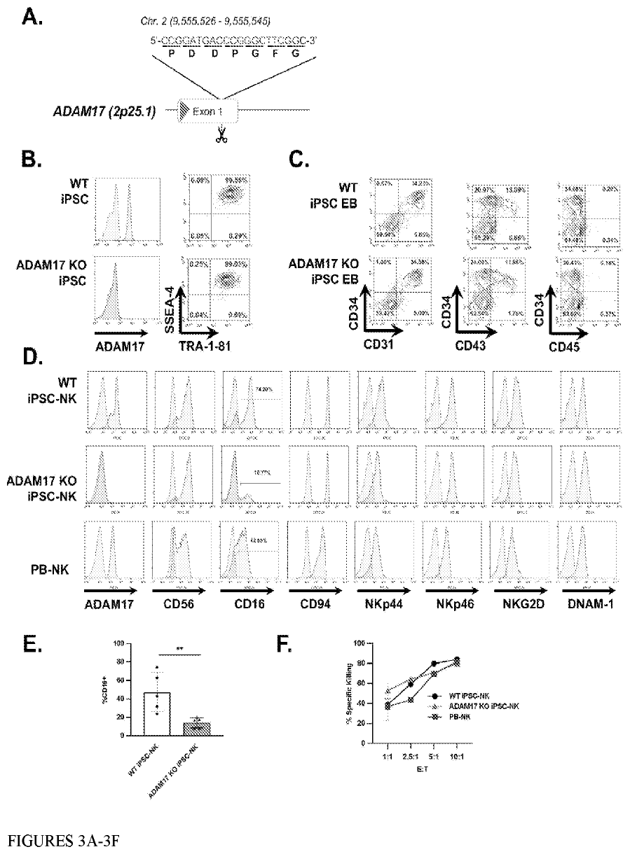

FIGS. 4A-4D show CD16-enriched ADAM17-deficient NK cells retain CD16, CD62L, and accumulate TNFα surface expression upon activation. FIG. 4A shows a schematic for the enrichment of CD16+NK cells. FIG. 4B shows CD16 enriched (as in (FIG. 4A)) WT, ADAM17 KO iPSC-NK and PB-NKs untreated, or stimulated with phorbol-12-myristate 13-acetate (80 nM) and ionomycin (130 μM) (PMA/Iono) for 4 hours, and assessed for CD16 surface expression. FIG. 4C shows quantification of % CD16+ cells relative to untreated, as in FIG. 4B. Each dot represents a separate experimental replicate. ***P<0.001, as determined by Student's two-tailed t-test assuming equal variances. Error bars shown indicate ±S.D. from experimental replicates. FIG. 4D shows CD16 enriched WT, ADAM17 KO iPSC-NK and PB-NKs untreated, or stimulated with PMA/Ionomycin for 4 hours, and assessed for TNFα and CD62L surface expression.

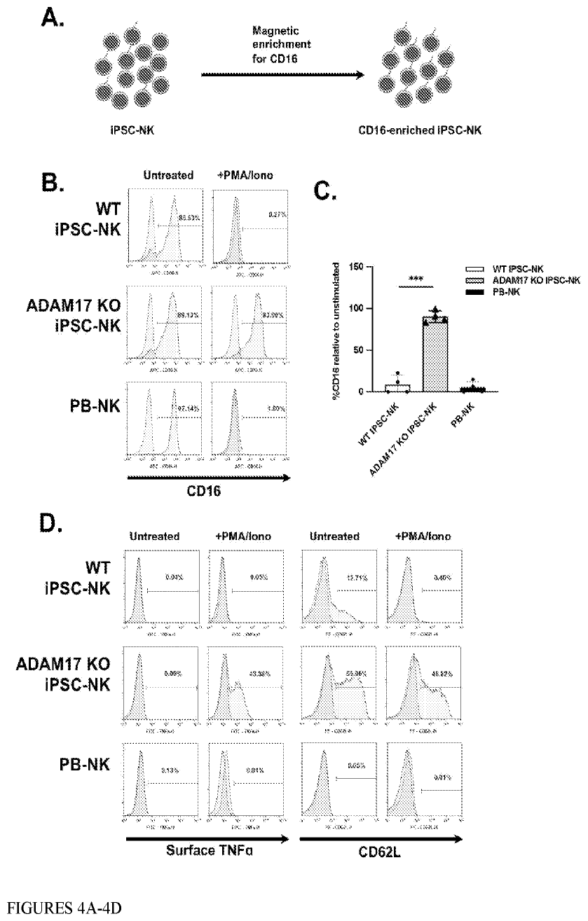

FIGS. 5A-5C show ADAM17-deficient iPSC-NK cells demonstrate enhanced antibody-derived cellular cytotoxicity (ADCC) activity expression. FIG. 5A shows CD107a vs. IFNγ and FIG. 5B shows TNFα vs. IFNγ on CD16-enriched WT, ADAM17 KO iPSC-NK and PB-NKs post 4-hour incubation with the indicated conditions. Co-culture conditions with tumor cells lines (K562, RAJI, CAL27) were incubated at 1:1 effector-to-target ratio, and plots shown are gated on CD56+ cells. Rituximab (anti-CD20) was used at final concentration of 10 μg/mL, and cetuximab (anti-EGFR) was used at final concentration of 1 μg/mL, and were compared to cells incubated with a human IgG1 isotype control at the same respective concentration. Intracellular staining was performed to assess IFNγ and TNFα levels post-fixation and permeabilization. FIG. 5C shows quantification of % CD107+(left), % IFNγ (middle), and % TNFα (right). Each dot for respective conditions indicates % positive cells analyzed from an independently performed experiment. *P<0.05 per Student's two-tailed t-test assuming equal variances. Error bars indicate ±S.D. from experimental replicate.

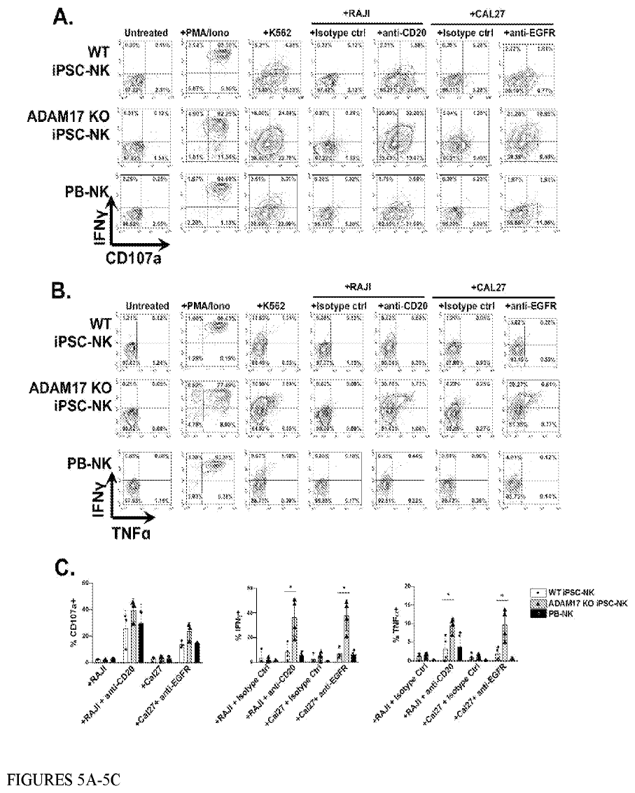

FIGS. 6A-6B show ADAM17-deficient iPSC-NK demonstrate enhanced cytotoxicity with therapeutic antibodies. FIG. 5A shows 24-hour cytotoxicity assay for CD16-enriched WT, ADAM17 KO iPSC-NKs and PB-NKs co-incubated with (A) RAJI cells with 10 μg/mL rituximab (anti-CD20) or human IgG1 isotype control, or FIG. 6B shows CAL27 cells with 1 μg/mL cetuximab (anti-EGFR) or human IgG1 isotype control. Cell death was detected as Caspase 3/7-positive events normalized to the number of intact cells (CellTrace Far Red). Data shown for each condition was normalized to background cell death at hour 0. Images were acquired a 1 hour intervals across 24 hours to assess changes in dead/live cell numbers using the IncuCyte platform. Error bars indicate ±S.E.M. of cell death calculated in 12 images taken across 3 wells per condition/timepoint.

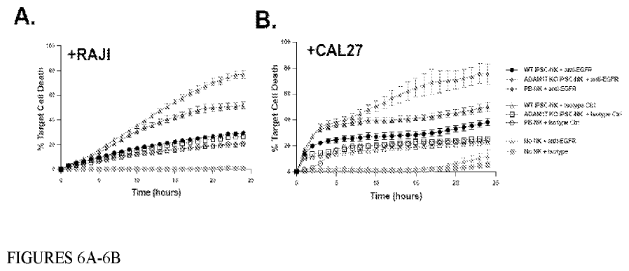

### DETAILED DESCRIPTION

The present disclosure relates, in part, to the surprising discoveries by the present inventors that engineered immune cells, ADAM17-deficient natural Killer (NK) cells, produced from stem cells have enhanced antibody-dependent cellular cytotoxicity (ADCC) and extended stable expression of CD16a, amongst other A-Disintegrin-And-Metalloprotease 17 substrates, as compared to non-engineered immune cells. The present disclosure provides engineered cells, compositions, and methods exploiting these discoveries.

In an aspect, the disclosure provides a novel genetic manipulation strategy to produce NK cells, such as induced pluripotent stem cell—natural killer (iPSC-NK) cells, with enhanced antibody-dependent cellular cytotoxicity (ADCC). ADCC is a key pathway that mediates NK cell cytotoxicity against antibody-opsonized target cells, and helps mediate therapeutic efficacy of anti-tumor antibodies. On NK cells, ADCC occurs via engagement of antibody-coated target cells with activating receptor FcγRIIIa, (CD16a) leading to proinflammatory cytokine upregulation, degranulation, and target cell death. Upon cellular activation, the CD16a ectodomain is cleaved from the NK cell surface by TNFα Converting Enzyme (TACE), also known as A-Disintegrin-And-Metalloprotease 17 (ADAM17). Cleavage of the ectodomain prevents further antibody binding and signaling through CD16a, which dampens NK cell activity. Therefore, NK cells lacking ADAM17 activity will be able to prevent CD16a shedding upon NK activation, and augment ADCC activity. Furthermore, nearly 80 ADAM17 substrates have been identified, most notably TNFβ and CD62L (L-selectin) that have also been noted to undergo cleavage upon NK cell activation and may affect cytotoxicity. ADAM17-deficient (ADAM17-KO) NK cells derived from iPSCs lacking ADAM17 expression are described herein. ADAM17-KO iPSC-NK cells have enhanced ADCC activity and more potent cytotoxicity against NK-resistant tumor cells in combination with therapeutic antibodies compared to unmodified wildtype iPSC-NK (WT iPSC-NK) and peripheral blood—natural killer (PB-NK) cell controls.

Various further aspects and embodiments of the disclosure are provided by the following description.

### Definitions

Prior to setting forth this disclosure in more detail, it may be helpful to an understanding thereof to provide definitions of certain terms to be used herein.

Unless defined otherwise, all technical and scientific terms used herein have the same meanings as commonly understood by one of ordinary skill in the art to which the present disclosure belongs. Any materials and methods similar or equivalent to those described herein can be used to practice the present invention. The practice of the present invention may employ conventional techniques of molecular biology (including recombinant techniques), microbiology, cell biology, biochemistry and immunology, which are within the skill of the art. Such techniques are explained fully in the literature, such as Molecular Cloning: A Laboratory Manual, second edition (Sambrook et al, 1989) Cold Spring Harbor Press; Oligonucleotide Synthesis (M J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C C. Blackwell, eds.); Gene Transfer Vectors for Mammalian Cells (J. M. Miller and M. P. Calos, eds., 1987); Current Protocols in Molecular Biology (F. M. Ausubel et al, eds., 1987); PCR: The Polymerase Chain Reaction, (Mullis et al, eds., 1994); Current Protocols in Immunology (J. E. Coligan et al, eds., 1991); Short Protocols in Molecular Biology (Wiley and Sons, 1999); Immunobiology (CA. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al, eds., J.B. Lippincott Company, 1993). Although any methods and materials similar or equivalent to those described herein can be used in the practice of the present invention, the exemplary methods, devices, and materials are described herein. For the purposes of the present disclosure, the following terms are defined below. Additional definitions are set forth throughout this disclosure.

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, an engineered immune cell, a pharmaceutical composition, and/or a method that “comprises” a list of elements (e.g., components, features, or steps) is not necessarily limited to only those elements (or components or steps), but may include other elements (or components or steps) not expressly listed or inherent to the engineered immune cell, pharmaceutical composition and/or method. Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It should be understood that the terms “a” and “an” as used herein refer to “one or more” of the enumerated components unless otherwise indicated. The use of the alternative (e.g., “or”) should be understood to mean either one, both, or any combination thereof of the alternatives.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment.

It will be further understood that there are a number of values disclosed therein, and that each value is also herein disclosed as “about” that particular value in addition to the value itself. In embodiments, “about” can be used to mean, for example, a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In various embodiments, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

All publications, patents, and patent applications mentioned in this specification are herein incorporated by reference to the same extent as if each individual publication, patent, or patent application was specifically and individually indicated to be incorporated by reference.

The section headings used herein are for organizational purposes only and are not to be construed as limiting the subject matter described.

### Engineered Cell

In an aspect, the disclosure provides an engineered immune cell, comprising an inactivating mutation in an endogenous A-Disintegrin-And-Metalloproteinase 17 (ADAM17) gene.

In some embodiments, the engineered immune cell is an induced pluripotent stem cell (iPSC)-derived immune cell. In some embodiments, the engineered immune cell is a peripheral blood (PB)-derived immune cell. In some embodiments, the engineered immune cell is a cord blood (CB)-derived immune cell.

As used herein, “induced pluripotent stem cell” or “iPSC cell” or “iPSCs” are used to refer to cells, derived from somatic cells, that have been reprogrammed back to an pluripotent state that are capable of proliferation, selectable differentiation, and maturation.

As used herein, “peripheral blood” or “peripheral blood cell” is used to refer to cells that originate from circulating blood and comprise hematopoietic stem cells that are capable of proliferation, selectable differentiation, and maturation.

As used herein, “cord blood cell” is used to refer to cells that originate from the umbilical cord and placenta and comprise hematopoietic stem cells that are capable of proliferation, selectable differentiation, and maturation.

In some embodiments, the engineered immune cell is a natural killer (NK) cell.

As used herein, and unless otherwise specified, a “natural killer cell” or “NK cell” is used to refer to cells that are cytotoxic lymphocytes that constitute a major component of the innate immune system. In humans a natural killer cell usually expresses the surface markers CD16 (FCyRIII) and CD56. NK cells are cytotoxic; small granules in cytoplasm that contain special proteins such as perforin and proteases known as granzymes. NK cells provide rapid responses to virally infected cells and respond to transformed cells. Upon release in close proximity to a cell slated for killing, perforin forms pores in the cell membrane of the target cell through which the granzymes and associated molecules can enter, inducing apoptosis. Thus, NK cells may act as effectors of lymphocyte population in anti-tumor and anti-infection immunity.

Typically, immune cells detect peptides from pathogens presented by Major Histocompatibility Complex (MHC) molecules on the surface of infected cells, triggering cytokine release, causing lysis or apoptosis. NK cells are unique, however, as they have the ability to recognize stressed cells regardless of whether peptides from pathogens are present on MHC molecules. They were named “natural killers” because of the initial notion that they do not require prior activation in order to kill a target. NK cells are large granular lymphocytes (LGL) and are known to differentiate and mature in the bone marrow from where they then enter into the circulation. In some embodiments, the NK cells are characterized by being CD56+CD3−. In some embodiments, the NK cells are characterized by being CD56+CD45+. In some embodiments, the NK cells are characterized by being CD56+CD45+CD3−. In some embodiments, the NK cells are characterized by being CD56+CD45+CD33−. In some embodiments, NK cells are characterized by being CD56+CD45+CD3− CD33−. In some embodiments, NK cells are characterized by being CD56+CD94+NKG2D+NKp44+NKp46+. In some embodiments, NK cells are characterized by being CD56+NKG2D+NKp44+NKp46+. In some embodiments, NK cells are characterized by being NKp30+NKp44+NKp46+. In some embodiments, NK cells are characterized by being NKp30+. In some embodiments, NK cells are characterized by being NKp44+. In some embodiments, NK cells are characterized by being NKp46+. In some embodiments, NK cells are characterized by being CD94+NKG2+. In some embodiments, NK cells are characterized by being inhibitory killer-immunoglobulin-like receptor (KIR+).

In some embodiments, the engineered immune cell is CD56+, CD94+, NKG2D+, NKp44+, and NKp46+.

As used herein, “engineered” or “genetically modified” or “transformed” are used interchangeably, wherein a cell has been manipulated by means of molecular reprogramming of a genomic sequence (e.g. by insertion, deletion, or substitution). Said cells include the primary transformed cell and progeny derived therefrom without regard to the number of passages. Progeny may not be completely identical in nucleic acid content to a parent cell and may contain mutations. Mutant progeny that have the same function or biological activity as screened or selected for in the originally transformed cell are included herein.

In some embodiments, the ADAM17 inactivating mutation is a knockout of an endogenous ADAM17 gene. In some embodiments, the engineered immune cell is a ADAM17−/− immune cell. In some embodiments, the ADAM17 inactivating mutation is a knockdown of an endogenous ADAM17 gene. In some embodiments, the cell expresses an exogenous polynucleotide that inhibits expression of an endogenous ADAM17 gene.

As used herein, “inactivating mutations” are used to describe mutations, i.e., insertions, deletions, or substitutions, of genomic nucleic acids that result in lack of formation of a transcript or translated product compared to that of a wildtype, or naturally occurring, genomic nucleic acid sequence. An inactivating mutation functionally inactivates, or renders non-functional and/or inoperative, a naturally occurring nucleic acid sequence for expression.

As used herein, a “knockout” or “KO” is used to refer to genetic manipulation, wherein the manipulation results in a gene being made nugatory and/or the function of the gene is eliminated, either mostly or completely. A knockout may be achieved through various methods known in the art, for example, integration of a premature stop codon or insertions and/or deletions to the degree of rendering the gene inoperative.

As used herein, a “knockdown” or “KD” is used to refer to genetic manipulation, wherein the manipulation results in a gene's expression being reduced. A knockdown may be achieved through use of genetic modification resulting in the reduced transcription of a gene or by use of introducing an exogenous polypeptide encoding a short DNA or RNA oligonucleotide(s) that have a sequence complementary to either the gene or an mRNA transcript resulting in lack of abundance of functional gene transcript.

The terms “exogenous” and “heterologous” are used herein to refer to any molecule, including nucleic acids, protein or peptides, small molecular compounds, and the like that originate from outside the organism. In contrast, the term “endogenous” refers to any molecule that originates from inside the organism (i.e., naturally produced by the organism).

As used herein, the term “ADAM17−/−” refers to a cell that has an inactivating mutations in both alleles of an endogenous ADAM17 gene, thereby making the cell a double knockout, wherein the cells exhibit absent, or near absent, expression for both genes.

The ability for immune cells to target and subsequently eliminate aberrant elements involves a myriad of potential devices. For example, one such device includes the use of antibody-dependent cellular cytotoxicity (ADCC), wherein immune cells mediate cell cytotoxicity against antibody-opsonized targets. Effector cells utilize ADCC by binding to target cells marked by specific antibodies bound to target antigens resulting in degranulation of the effector cell releasing cytotoxic factors rendering the target dead. Fc receptors (activating receptors) on the surface of effector cells bind to the Fc region of the antibody bound to an antigen stimulating ADCC.

Extracellular domains of Fc receptors are subject to degradation by enzymes after cellular activation. For example, CD16 receptors have been identified as one of many tentative substrates for the enzyme A-Disintegrin-And-Metalloprotease-17 (ADAM17). Disruption of effector cell CD16 receptors would render the cells incapable of antibody binding and signaling resulting in reduced ADCC.

In some embodiments, the engineered immune cell comprises a Fc receptor. In some embodiments, the Fc receptor remains fully intact or nondegraded after activation. In some embodiments, the Fc receptor is a FcγRIII (CD16) receptor. In some embodiments, the CD16 receptor is FcÎ³ RIIIa (CD16a). In some embodiments, the CD16 receptor is FcγRIIIb (CD16b). In some embodiments, the CD16 receptor is CD16a and/or CD16b. In some embodiments, the Fc receptor is FcεRI.

The engineered immune cells may exhibit upregulation and/or stabilization of certain cell surface markers compared to wildtype cell counterparts. In some embodiments, the engineered immune cells exhibit stabilization of CD16 compared to wildtype cell counterparts. In some embodiments, the engineered immune cells exhibit stabilization of CD62L compared to wild type counterparts. In some embodiments, the engineered immune cells exhibit enhanced surface expression of TNFα compared to wild type counterparts.

In some embodiments, the engineered immune cell has increased expression of CD16a, TNF-α, and CD62L (L-selectin) as compared to a non-engineered immune cell after stimulation with a stimulating agent.

Stimulating agents include molecules that trigger activation of an NK cell. Exemplary types of stimulating agents include, but are not limited to, molecules capable of triggering CD16 or phorbol esters.

In some embodiments, expression of CD16a in the engineered immune cell is stably maintained for a period of at least about 6 weeks during expansion in a culture.

In some embodiments, the immune cell is a human immune cell.

### Purified Cell Composition

In an aspect, the disclosure provides a purified cell composition comprising one or more of the engineered immune cell of the disclosure.

As used herein, a composition containing a “purified cell population” or “purified cell composition” means that at least 30%, 50%, 60%, typically at least 70%, and more preferably 80%, 90%, 95%, 98%, 99%, or more of the cells in the composition are of the identified type.

In some embodiments, at least about 50%, 60%, 70%, 80%, 90%, or 100% of engineered immune cells express tumor necrosis factor-alpha (TNF-α) after stimulation with a stimulating agent. In some embodiments, at least about 50% of engineered immune cells express tumor necrosis factor-alpha (TNF-α) after stimulation with a stimulating agent. In some embodiments, at least about 60% of engineered immune cells express tumor necrosis factor-alpha (TNF-α) after stimulation with a stimulating agent. In some embodiments, at least about 70% of engineered immune cells express tumor necrosis factor-alpha (TNF-α) after stimulation with a stimulating agent. In some embodiments, at least about 80% of engineered immune cells express tumor necrosis factor-alpha (TNF-α) after stimulation with a stimulating agent. In some embodiments, at least about 90% of engineered immune cells express tumor necrosis factor-alpha (TNF-α) after stimulation with a stimulating agent. In some embodiments, at least about 100% of engineered immune cells express tumor necrosis factor-alpha (TNF-α) after stimulation with a stimulating agent. In some embodiments, about 71% of engineered immune cells express tumor necrosis factor-alpha (TNF-α) after stimulation with a stimulating agent. In some embodiments, at least about 71% of engineered immune cells express tumor necrosis factor-alpha (TNF-α) after stimulation with a stimulating agent. In some embodiments, at least about 10%, 20%, 30%, 40%, 50%, 60%, 70%, 80%, 90%, or 100% of engineered immune cells express CD62L (L-selectin) after stimulation with a stimulating agent. In some embodiments, at least about 30% of engineered immune cells express CD62L (L-selectin) after stimulation with a stimulating agent. In some embodiments, at least about 40% of engineered immune cells express CD62L (L-selectin) after stimulation with a stimulating agent. In some embodiments, at least about 50% of engineered immune cells express CD62L (L-selectin) after stimulation with a stimulating agent. In some embodiments, at least about 60% of engineered immune cells express CD62L (L-selectin) after stimulation with a stimulating agent. In some embodiments, at least about 70% of engineered immune cells express CD62L (L-selectin) after stimulation with a stimulating agent. In some embodiments, at least about 80% of engineered immune cells express CD62L (L-selectin) after stimulation with a stimulating agent. In some embodiments, at least about 90% of engineered immune cells express CD62L (L-selectin) after stimulation with a stimulating agent. In some embodiments, at least about 100% of engineered immune cells express CD62L (L-selectin) after stimulation with a stimulating agent. In some embodiments, at about 42% of engineered immune cells express CD62L (L-selectin) after stimulation with a stimulating agent. In some embodiments, at least about 42% of engineered immune cells express CD62L (L-selectin) after stimulation with a stimulating agent.

In some embodiments, the engineered immune cells have increased expression of CD107a and interferon gamma (IFNγ) as compared to non-engineered immune cells after co-incubation with a disease cell line and an antibody specific to said disease cell line. In some embodiments the disease cell line is a cell line comprising a malignancy. In some embodiments the disease cell line is a cell line comprising a viral infection.

In some embodiments, disease cell line is a B-lymphoma cell line and the antibody specific to said disease cell line is an anti-CD20 antibody. In some embodiments, the anti-CD20 antibody is rituximab. In some embodiments, the engineered immune cells exhibit at least about 10%, 20%, 30%, 40%, 50%, 60%, 70%, 80%, 90%, or 100% increased expression of CD107a. In some embodiments, the engineered immune cells exhibit at least about 20% increased expression of CD107a. In some embodiments, the engineered immune cells exhibit at least about 30% increased expression of CD107a. In some embodiments, the engineered immune cells exhibit at least about 40% increased expression of CD107a. In some embodiments, the engineered immune cells exhibit at least about 50% increased expression of CD107a. In some embodiments, the engineered immune cells exhibit at least about 60% increased expression of CD107a. In some embodiments, the engineered immune cells exhibit at least about 70% increased expression of CD107a. In some embodiments, the engineered immune cells exhibit at least about 80% increased expression of CD107a. In some embodiments, the engineered immune cells exhibit at least about 90% increased expression of CD107a. In some embodiments, the engineered immune cells exhibit at least about 100% increased expression of CD107a. In some embodiments, the engineered immune cells exhibit about 45% increased expression of CD107a. In some embodiments, the engineered immune cells exhibit at least about 45% increased expression of CD107a. In some embodiments, the engineered immune cells exhibit at least about 10%, 20%, 30%, 40%, 50%, 60%, 70%, 80%, 90%, or 100% increased expression of IFNγ. In some embodiments, the engineered immune cells exhibit at least about 10% increased expression of IFNγ. In some embodiments, the engineered immune cells exhibit at least about 20% increased expression of IFNγ. In some embodiments, the engineered immune cells exhibit at least about 30% increased expression of IFNγ. In some embodiments, the engineered immune cells exhibit at least about 40% increased expression of IFNγ. In some embodiments, the engineered immune cells exhibit at least about 50% increased expression of IFNγ. In some embodiments, the engineered immune cells exhibit at least about 60%, increased expression of IFNγ. In some embodiments, the engineered immune cells exhibit at least about 70% increased expression of IFNγ. In some embodiments, the engineered immune cells exhibit at least about 80% increased expression of IFNγ. In some embodiments, the engineered immune cells exhibit at least about 90% increased expression of IFNγ. In some embodiments, the engineered immune cells exhibit at least about 100% increased expression of IFNγ. In some embodiments, the engineered immune cells exhibit about 36% increased expression of IFNγ. In some embodiments, the engineered immune cells exhibit at least about 36% increased expression of IFNγ.

In some embodiments, the disease cell line is a squamous cell carcinoma cell line and the antibody specific to said disease cell line is an anti-EGFR antibody. In some embodiments, the anti-EGFR antibody is cetuximab. In some embodiments, the engineered immune cells exhibit at least about 10%, 20%, 30%, 40%, 50%, 60%, 70%, 80%, 90%, or 100% increased expression of CD107a. In some embodiments, the engineered immune cells exhibit at least about 10% increased expression of CD107a. In some embodiments, the engineered immune cells exhibit at least about 20% increased expression of CD107a. In some embodiments, the engineered immune cells exhibit at least about 30% increased expression of CD107a. In some embodiments, the engineered immune cells exhibit at least about 40% increased expression of CD107a. In some embodiments, the engineered immune cells exhibit at least about 50% increased expression of CD107a. In some embodiments, the engineered immune cells exhibit at least about 60% increased expression of CD107a. In some embodiments, the engineered immune cells exhibit at least about 70% increased expression of CD107a. In some embodiments, the engineered immune cells exhibit at least about 80% increased expression of CD107a. In some embodiments, the engineered immune cells exhibit at least about 90% increased expression of CD107a. In some embodiments, the engineered immune cells exhibit at least about 100% increased expression of CD107a. In some embodiments, the engineered immune cells exhibit about 29% increased expression of CD107a. In some embodiments, the engineered immune cells exhibit at least about 29% increased expression of CD107a. In some embodiments, the engineered immune cells exhibit at least about 10%, 20%, 30%, 40%, 50%, 60%, 70%, 80%, 90%, or 100% increased expression of IFNγ. In some embodiments, the engineered immune cells exhibit at least about 10% increased expression of IFNγ. In some embodiments, the engineered immune cells exhibit at least about 20% increased expression of IFNγ. In some embodiments, the engineered immune cells exhibit at least about 30% increased expression of IFNγ. In some embodiments, the engineered immune cells exhibit at least about 40% increased expression of IFNγ. In some embodiments, the engineered immune cells exhibit at least about 50% increased expression of IFNγ. In some embodiments, the engineered immune cells exhibit at least about 60% increased expression of IFNγ. In some embodiments, the engineered immune cells exhibit at least about 70% increased expression of IFNγ. In some embodiments, the engineered immune cells exhibit at least about 80% increased expression of IFNγ. In some embodiments, the engineered immune cells exhibit at least about 90% increased expression of IFNγ. In some embodiments, the engineered immune cells exhibit at least about 100% increased expression of IFNγ. In some embodiments, the engineered immune cells exhibit about 39% increased expression of IFNγ. In some embodiments, the engineered immune cells exhibit at least about 39% increased expression of IFNγ.

In some embodiments, the engineered immune cells exhibit enhanced antibody-dependent cellular cytotoxicity (ADCC) as compared to non-engineered immune cells. In some embodiments, the engineered immune cells are capable of at least about 50%, 60%, 70%, 80%, 90%, or 100% targeted cell death, wherein the immune cells of the disclosure are in the presence of an antigen-specific antibody specific to a targeted cell. In some embodiments, the engineered immune cells are capable of at least about 50% targeted cell death, wherein the immune cells of the disclosure are in the presence of an antigen-specific antibody specific to a targeted cell. In some embodiments, the engineered immune cells are capable of at least about 60% targeted cell death, wherein the immune cells of the disclosure are in the presence of an antigen-specific antibody specific to a targeted cell. In some embodiments, the engineered immune cells are capable of at least about 70% targeted cell death, wherein the immune cells of the disclosure are in the presence of an antigen-specific antibody specific to a targeted cell. In some embodiments, the engineered immune cells are capable of at least about 80% targeted cell death, wherein the immune cells of the disclosure are in the presence of an antigen-specific antibody specific to a targeted cell. In some embodiments, the engineered immune cells are capable of at least about 90% targeted cell death, wherein the immune cells of the disclosure are in the presence of an antigen-specific antibody specific to a targeted cell. In some embodiments, the engineered immune cells are capable of at least about 100% targeted cell death, wherein the immune cells of the disclosure are in the presence of an antigen-specific antibody specific to a targeted cell. In some embodiments, the engineered immune cells are capable of about 80% targeted cell death, wherein the immune cells of the disclosure are in the presence of an antigen-specific antibody.

ADAM17-KO iPSCs successfully differentiate into hematopoietic progenitor cells, then to NK cells that uniformly express typical NK cell surface markers including CD56, CD94, NKG2D, NKp44, and NKp46. ADAM17-KO iPSC-derived NK cells are functional and kill K562 erythroleukemia cells comparable to wildtype iPSC-derived NK cells (WT iPSC-NK cells) and healthy donor-derived peripheral blood NK cells (PB-NK cells) in vitro. Surprisingly, upon differentiation, ADAM17-KO iPSC-NK cells express −20% lower CD16a surface expression compared to WT iPSC-NK cells, but stably retain CD16a expression after enrichment for CD16a+ cells and over 6 weeks of expansion in culture. WT iPSC-NK cells and PB-NK cells rapidly lose CD16a surface expression upon stimulation with phorbol esters, while ADAM17 KO iPSC-NK cells maintain over 90% CD16a expression after this stimulation. Additionally, a significantly higher proportion of ADAM17-KO iPSCs express TNF-α (71%) and CD62L (L-Selectin) (36%)—two other known ADAM17 substrates, on the cell surface after stimulation with phorbol esters for 4 hours compared to WT iPSC-NK (7% TNF-α+, 2% L-Selectin+) and PB-NK (2% TNF-α+, 1% L-Selectin+). CD16a+ADAM17-KO iPSC-NK cells mediate increased CD107a (45%) and IFNγ (39%) expression when co-incubated with RAJI B-lymphoma cells in the presence of the anti-CD20 antibody rituximab, compared to CD16a+WT iPSC-NK (32% CD107a+, 11% IFNγ) and PB-NK (37% CD107a+, 7% IFNγ) cells. Similarly, CD16a+ADAM17-KO iPSC-NK cells upregulate increased CD107a (29%) and IFNγ (42%) expression when co-incubated with CAL27 squamous cell carcinoma cells in the presence of the anti-EGFR antibody cetuximab, compared to CD16a+WT iPSC-NK (12% CD107a+, 8% IFNγ) and PB-NK (14% CD107a+, 6% IFNγ). Long-term (24 hour) cytotoxicity assay against RAJI cells in the presence of rituximab demonstrates higher cytotoxicity in CD16a+ADAM17-KO iPSC-NK cells compared to CD16a+WT iPSC-NK and CD16a+PB-NK cells over time.

The enhanced attributes of the engineered immune cell(s) of the disclosure compared to non-engineered immune cells are provided in the Table 1 below.

TABLE 1

| | | | |
|---|---|---|---|
| | Donor-derived peripheral | Wildtype induced | Engineered induced |
| | blood cells | pluripotent stem cells | pluripotent stem cells |
| Marker | (PB-NK) | (WT iPSC-NK) | (ADAM17-KO iPSC-NK) |

Length of time of stable expression after enrichment for CD16a+ in expansion culture

| | |
|---|---|
| CD16a | 6 weeks+ |

Percentage of cells expressing markers after stimulation with phorbol esters

| | | | |
|---|---|---|---|
| CD16a | Rapid loss | Rapid loss | 90% maintain |

Percentage of cells expressing markers after stimulation with phorbol esters for 4 hours

| | | | |
|---|---|---|---|
| TNFα | 2% | 7% | 71% |
| CD62L | 1% | 2% | 36% |

Percentage of increased expression of markers after co-incubation with diseased cells (B-lymphoma cells)
and antibodies specific to diseased cells (anti-CD20 antibody - rituximab)

| | | | |
|---|---|---|---|
| CD 107a | 37% | 32% | 45% |
| IFNγ | 7% | 11% | 39% |

Percentage of increased expression of markers after co-incubation with diseased cells (squamous cell
carcinoma cells) and antibodies specific to diseased cells (anti-EGFR antibody - cetuximab)

| | | | |
|---|---|---|---|
| CD107a | 14% | 12% | 29% |
| IFNγ | 6% | 8% | 42% |

Together, this demonstrates that ADAM17-KO iPSC-NK cells derived from a renewable source of gene-edited iPSCs possess enhanced ADCC potential, and provide a promising candidate to be used for standardized, off-the-shelf NK cell-based therapies in conjunction with therapeutic antibodies.

### Method of Making

The disclosure provides a method of making a NK cell culture as described herein, comprising genetically modifying a cell to be ADAM17-deficient.

As used herein, and unless otherwise specified, “ADAM17 deficient” or “ADAM17-deficient” means the lack of at least a part of a naturally occurring nucleic acid sequence for expression of the ADAM17 gene, as compared to the ADAM17 gene expression in a naturally occurring, or wildtype, immune cell. Such deficiencies may be the result of inactivating mutations as disclosed herein.

In an aspect, the disclosure provides a method of making the engineered immune cell of the disclosure comprising: a) introducing an inactivation mutation in an endogenous A-Disintegrin-And-Metalloproteinase 17 (ADAM17) gene into a stem cell; and b) differentiating the stem cell into an immune cell. In some embodiments, the stem cell is an induced pluripotent stem cell (iPSC), a peripheral blood cell, or a cord blood cell. In some embodiments, the immune cell is a natural killer cell (NK) cell.

In some embodiments, the method of making the engineered immune cell of the disclosure comprising: a) differentiating a stem cell into an immune cell; and b) introducing an inactivation mutation in an endogenous A-Disintegrin-And-Metalloproteinase 17 (ADAM17) gene into said immune cell. In some embodiments, the stem cell is an introduced pluripotent stem cell (iPSC), a peripheral blood cell, or a cord blood cell. In some embodiments, the immune cell is a natural killer cell (NK) cell.

Genome editing tools may be used to engineer and/or manipulate cells. In some embodiments, the immune cell of the disclosure may be engineered with either CRISPR, TALEN, or ZFN genome editing tools.

Genome editing tools such as the clustered regularly interspaced short palindromic repeats (CRISPR) system may be used to genetically modify cells. CRISPR can be used in a wide variety of organisms (e. g., used to add, disrupt, or change the sequence of specific genes). “CRISPR” or “CRISPR gene editing” as used herein refers to a set of clustered regularly interspaced short palindromic repeats, or a system comprising such a set of repeats. “Cas”, as used herein, refers to a CRISPR-associated protein. A “CRISPR/Cas” system refers to a system derived from CRISPR and Cas which can be used to silence, knock out, or mutate a target gene.

The CRISPR/Cas system is based on two elements. The first element is an endonuclease, or Cas, (e.g., Cas9 and MAD7) that has a binding site for the second element, which is the guide polynucleotide (e.g., guide RNA or gRNA). The guide polynucleotide (e.g., guide RNA) directs the Cas protein to double stranded DNA templates based on sequence homology. The Cas protein then cleaves that DNA template. By delivering the Cas protein and appropriate guide polynucleotides (e.g., guide RNAs) into a cell, the organism's genome is cut at a desired location. Following cleavage of a targeted genomic sequence by a Cas/gRNA complex, one of two alternative DNA repair mechanisms can restore chromosomal integrity: 1) non-homologous end joining (NHEJ) which generates insertions and/or deletions of a few base-pairs (bp) of DNA at the gRNA cut site, or 2) homology-directed repair (HDR) which can correct the lesion via an additional “bridging” DNA template that spans the gRNA cut site. CRISPR/Cas systems are classified by class and by type. Class 2 systems currently represent a single interference protein that is categorized into three distinct types (types II, V and VI). Any class 2 CRISPR/Cas system suitable for gene editing, for example a type II, a type V or a type VI system, is envisaged as within the scope of the instant disclosure. Exemplary Class 2 type II CRISPR systems include Cas9, Csn2 and Cas4. Exemplary Class 2, type V CRISPR systems include, Cas12, Cas12a (Cpf1), Cas12b (C2c1), Cas12c (C2c3), Cas12d (CasY), Cas12e (CasX), Cas12f, Cas12g, Cas12h, Cas12i and Cas12k (C2c5). Exemplary Class 2 Type VI systems include Cas13, Cas13a (C2c2) Cas13b, Cas13c and Cas13d.

The CRISPR sequence, sometimes called a CRISPR locus, comprises alternating repeats and spacers. In a naturally-occurring CRISPR, the spacers usually comprise sequences foreign to the bacterium such as a plasmid or phage sequence. As described herein, spacer sequences may also be referred to as “targeting sequences.” In CRISPR/Cas systems for a genetic engineering, the spacers are derived from the target gene sequence (the gNA).

The targeting sequence can be designed or chosen using computer programs known to persons of ordinary skill in the art. The computer program can use variables, such as predicted melting temperature, secondary structure formation, predicted annealing temperature, sequence identity, genomic context, chromatin accessibility, % GC, frequency of genomic occurrence (e.g., of sequences that are identical or are similar but vary in one or more spots as a result of mismatch, insertion or deletion), methylation status, presence of SNPs, and the like. Available computer programs can take as input NCBI gene IDs, official gene symbols, Ensembl Gene IDs, genomic coordinates, or DNA sequences, and create an output file containing sgRNAs targeting the appropriate genomic regions designated as input. The computer program may also provide a summary of statistics and scores indicating on- and off-target binding of the sgRNA for the target gene (Doench et al. Nat Biotechnol. 34:184-191 (2016)).

The target sequence is complementary to, and hybridizes with, the targeting sequence of the gRNA. The target nucleic acid sequence can comprise 20 nucleotides. The target nucleic acid can comprise less than 20 nucleotides. The target nucleic acid can comprise more than 20 nucleotides. The target nucleic acid can comprise at least: 5, 10, 15, 16, 17, 18, 19, 20, 21, 22, 23, 24, 25, 30 or more nucleotides. The target nucleic acid can comprise at most: 5, 10, 15, 16, 17, 18, 19, 20, 21, 22, 23, 24, 25, 30 or more nucleotides.

The CRISPR/Cas system can thus be used to edit a target gene, such as a gene targeted for editing in the cells described herein, by adding or deleting a base pair, introducing a premature stop codon, or introducing a frame-shift mutation which thus decreases expression of the target, in part or completely. The CRISPR/Cas system can alternatively be used like RNA interference, turning off a target gene in a reversible fashion. In a mammalian cell, for example, the RNA can guide the Cas protein to a target gene promoter, sterically blocking RNA polymerases.

Further aspects of the CRISPR/Cas system known to those of ordinary skill are described in PCT Publication Nos. WO 2017/049266 and WO 2017/223538, the entire contents of which are hereby incorporated by reference. These and other well-known and new techniques, such as TALEN and Zinc Finger Nucleases, for generating NK cells of the present disclosure are contemplated by the present invention.

In some embodiments, the engineered NK cells described herein are edited using TALEN gene editing. “TALEN” or “TALEN gene editing” refers to a transcription activator-like effector nuclease, which is an artificial nuclease used to edit a target gene. TALENs are produced artificially by fusing a TAL effector DNA binding domain to a DNA cleavage domain. Transcription activator-like effectors (TALEs) can be engineered to bind any desired DNA sequence, including a portion of target genes such as TCR subunits, MHC class I complex components, or CD52. By combining an engineered TALE with a DNA cleavage domain, a restriction enzyme can be produced which is specific to any desired DNA sequence, including a target gene sequence. These can then be introduced into a cell, wherein they can be used for genome editing. Boch (2011) Nature Biotech. 29: 135-6; and Boch et al. (2009) Science 326: 1509-12; Moscou et al. (2009) Science 326: 3501.

In some embodiments, the engineered NK cells described herein are edited using ZFN gene editing. “ZFN” or “Zinc Finger Nuclease” or “ZFN gene editing” refer to a zinc finger nuclease, an artificial nuclease which can be used to edit a target gene. Like a TALEN, a ZFN comprises a Fold nuclease domain (or derivative thereof) fused to a DNA-binding domain. In the case of a ZFN, the DNA-binding domain comprises one or more zinc fingers. Carroll et al. (2011) Genetics Society of America 188: 773-782; and Kim et al. (1996) Proc. Natl. Acad. Sci. USA 93: 1156-1160.

The immune cells described herein can be modified using methods known in the art. The various gene editing systems described herein may be used to modify the immune cell to delete, inactivate, reduce expression, or otherwise inhibit function of a target gene or a target gene product.

A cell, such as a NK cell or a iPSC can be genetically modified to be ADAM17 deficient, or ADAM17 knockout (ADAM17 KO) by any known or discovered gene editing method, such as by CRISPR, TALEN, or ZFN techniques. In some embodiments, a NK cell is genetically modified to be ADAM17 deficient, or ADAM17 knockout (ADAM17KO) by a gene editing method. In some embodiments, an iPSC is genetically modified to be ADAM17 deficient, or ADAM17 Knock-Out (ADAM17KO) by a gene editing method. In some embodiments, a NK cell is genetically modified to be ADAM17 deficient, or ADAM17 knockdown (ADAM17KD) by a gene editing method. In some embodiments, an iPSC is genetically modified to be ADAM17 deficient, or ADAM17-knockdown (ADAM17KD) by a gene editing method.

In some embodiments, the disclosure provides a method of making an engineered immune cell comprising: a) transducing an iPSC with ADAM17 gRNA and MAD7 constructs wherein an inactivating mutation in an endogenous ADAM17 gene occurs in both alleles, thereby generating a ADAM17−/− iPSC; and b) differentiating the ADAM17−/− iPSC into a NK cell, thereby generating an ADAM17−/− NK cell.

The term “nucleic acid” or “polynucleotide”, includes DNA and RNA such as genomic DNA, cDNA and mRNA, or combinations thereof. The nucleic acid may comprise, in addition to the sequence enabling the genetic modifications of the disclosure, further sequences such as those required for the transcription and/or translation of the nucleic acid enabling said genetic modifications. This may include a promoter, enhancer, transcription and/or translation initiation and/or termination sequences, selection markers, sequences protecting or directing the RNA and/or enabling the genetic modifications within the cell. The selection and combination of these sequences is within the knowledge of the person skilled in the art and may be selected in accordance with the cell the nucleic acid is intended for.

Polynucleotides enabling the genetic modifications of the disclosure may be delivered to cells as an isolated nucleic acid or in a vector. The isolated nucleic acid or the vector may be delivered in lipid- or lipid-based delivery system, such as a liposome. Alternatively, the vector may comprise viral proteins, such as when the vector is a viral vector. The term “vector” as used herein refers to a construction comprised of genetic material designed to direct transformation or transductions of a targeted cell. A vector contains multiple genetic elements positionally and sequentially oriented with other necessary elements such that the nucleic acid in a nucleic acid cassette can be transcribed and when necessary translated in the transfected cells. The term vector as used herein can refer to nucleic acid, e.g., DNA derived from a plasmid, cosmid, phagemid, bacteriophage, virus, retrovirus, adenovirus, adeno-associated virus, lentivirus, or other type of virus into which one or more fragments of nucleic acid may be inserted or cloned which encode for particular proteins. The term “plasmid” as used herein refers to a construction comprised of extrachromosomal genetic material, usually of a circular duplex of DNA which can replicate independently of chromosomal DNA. The plasmid does not necessarily replicate.

Any suitable vectors are envisaged as within the scope of the instant disclosure. The polynucleotides enabling the genetic modifications of the disclosure can be cloned into a number of types of vectors. For example, the polynucleotides enabling the genetic modifications of the disclosure may be cloned into a vector including, but not limited to a plasmid, a phagemid, a phage derivative, an animal virus, and a cosmid. Vectors of particular interest include expression vectors, replication vectors, probe generation vectors, and sequencing vectors. Expression vectors may be provided to cells, such as immune cells, in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

The purpose of the vector is to provide a nucleic acid sequence in cells or tissue. Expression includes the efficient transcription of an inserted gene or nucleic acid sequence. Expression products may be proteins, polypeptides, or RNA. The nucleic acid sequence can be contained in a nucleic acid cassette. Expression of the nucleic acid can be continuous, constitutive, or regulated. The vector can also be used as a prokaryotic element for replication of plasmid in bacteria and selection for maintenance of plasmid in bacteria.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). One method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In some embodiments, transducing comprises either calcium phosphate-mediated gene transfer, DEAE-dextran-mediated gene transfer, liposome-mediated gene transfer, electroporation-mediated gene transfer, viral vector-mediated gene transfer, or nucleofection-mediated gene transfer. In some embodiments, transducing is accomplished by calcium phosphate-mediated gene transfer. In some embodiments, transducing is accomplished by liposome-mediated gene transfer. In some embodiments, transducing is accomplished by electroporation-mediated gene transfer. In some embodiments, transducing is accomplished by viral vector-mediated gene transfer. In some embodiments, transducing is accomplished by nucleofection-mediated gene transfer.

Regardless of the method used to introduce exogenous nucleic acids into a host cell, in order to confirm the presence of the recombinant DNA sequence in the host cell, or confirm effect of genomic modulation, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or other assays.

Generally, techniques for differentiating an induced pluripotent cell involve modulation of specific cellular pathways, either directly or indirectly, using polynucleotide-, polypeptide- and/or small molecule-based approaches. The developmental potency of a cell may be modulated, for example, by contacting a cell with one or more modulators. “Contacting”, as used herein, can involve culturing cells in the presence of one or more factors (such as, for example, small molecules, proteins, peptides, etc.). In some embodiments, a cell is contacted with one or more agents to induce cell differentiation. Such contact, may occur for example, by introducing the one or more agents to the cell during in vitro culture. Thus, contact may occur by introducing the one or more agents to the cell in a nutrient cell culture medium. The cell may be maintained in the culture medium comprising one or more agents for a period sufficient for the cell to achieve the differentiation phenotype that is desired.

Differentiation of stem cells requires a change in the culture system, such as changing the stimuli agents in the culture medium or the physical state of the cells. A conventional strategy utilizes the formation of embryoid bodies (EBs) as a common and critical intermediate to initiate the lineage-specific differentiation. EBs are three-dimensional clusters that have been shown to mimic embryo development as they give rise to numerous lineages within their three-dimensional area. Through the differentiation process simple EBs (for example, aggregated pluripotent stem cells elicited to differentiate) continue maturation and develop into a cystic EB at which time, they are further processed to continue differentiation. EB formation is initiated by bringing pluripotent stem cells into close proximity with one another in three-dimensional multilayered clusters of cells. Typically, this is achieved by one of several methods including allowing pluripotent cells to sediment in liquid droplets, sedimenting cells into “U” bottomed well-plates or by mechanical agitation. To promote EB development, the pluripotent stem cell aggregates require further differentiation cues, as aggregates maintained in pluripotent culture maintenance medium do not form proper EBs. This may be followed by additional stimulation differentiating the iPSCs to hematopoietic cells and then to convert the hematopoietic progenitor cells into natural killer (NK).

Illustrative methods for making and using engineered cells are provided in Int'l Pat. Appl. Nos. WO 2013/163171 A1, WO 2017/078807 A1, and WO 2018/147801 the disclosures of which are incorporated by reference herein in their entireties.

As used herein, “differentiate” or “differentiated” are used to refer to the process and conditions by which immature (unspecialized) cells acquire characteristics becoming mature (specialized) cells thereby acquiring particular form and function. Stem cells (unspecialized) are often exposed to varying conditions (e.g., growth factors and morphogenic factors) to induce specified lineage commitment, or differentiation, of said stem cells. The process by which an unspecialized (“uncommitted”) or less specialized cell acquires the features of a specialized cell such as, for example, a blood cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type.

Differentiation marker gene(s) may be monitored to gauge a cells state of differentiation. As used herein, the term “differentiation marker gene,” or “differentiation gene,” refers to genes whose expression are indicative of cell differentiation occurring within a cell, such as a pluripotent cell. Differentiation marker genes include, but are not limited to, the following genes: FOXA2, FGF5, SOX17, XIST, NODAL, COL3A1, OTX2, DUSP6, EOMES, NR2F2, NR0B1, CXCR4, CYP2B6, GATA3, GATA4, ERBB4, GATA6, HOXC6, INHA, SMAD6, RORA, NIPBL, TNFSF11, CDH11, ZIC4, GAL, SOX3, PITX2, APOA2, CXCLS, CER1, FOXQ1, MLL5, DPP10, GSC, PCDH10, CTCFL, PCDH20, TSHZ1, MEGF10, MYC, DKK1, BMP2, LEFTY2, HES1, CDX2, GNAS, EGR1, COL3A1, TCF4, HEPH, KDR, TOX, FOXA1, LCK, PCDH7, CD1D FOXG1, LEFTY1, TUJ1, T gene (Brachyury), ZIC1, GATA1, GATA2, HDAC4, HDAC5, HDAC7, HDAC9, NOTCH1, NOTCH2, NOTCH4, PAX5, RBPJ, RUNX1, STAT1, STAT3 and STATS.

“Culture” or “cell culture” refers to the maintenance, growth and/or differentiation of cells in an in vitro environment. “Cell culture media,” “culture media” (singular “medium” in each case), “supplement” and “media supplement” refer to nutritive compositions that cultivate cell cultures.

“Cultivate,” or “maintain,” refers to the sustaining, propagating (growing) and/or differentiating of cells outside of tissue or the body, for example in a sterile plastic (or coated plastic) cell culture dish or flask. “Cultivation,” or “maintaining,” may utilize a culture medium as a source of nutrients, hormones and/or other factors helpful to propagate and/or sustain the cells.

Multipotent hematopoietic stem cells provide the basis of two major progenitor cell lineages. The first cell lineage is the common lymphoid progenitor cell lineage, wherein a multipotent hematopoietic stem cell (hemocytoblast) differentiates into a lymphoid progenitor cell, which has the capability to further differentiate into a natural killer cell, T lymphocyte, or B lymphocyte; or differentiate even further from a B lymphocyte to a plasma cell. The other major cell lineage is the common myeloid progenitor cell lineage, wherein a hemocytoblast differentiates into a myeloid progenitor cell, which has the capability to further differentiate into a megakaryocyte, erythrocyte, platelet, mast cell, or myeloblast; or differentiate even further from a myeloblast to a basophil, neutrophil, eosinophil, or monocyte; or yet further differentiate from a monocyte to a macrophage.

As used herein, the term “pluripotent” refers to the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. As such, the term “pluripotent stem cell”, as used herein, refers to a subset of undifferentiated cells that are capable of giving rise to hematopoietic stem and progenitor cells via hematopoietic transition.

The term “hematopoietic stem cell,” as used herein, refers to CD34+ stem cells capable of giving rise to both mature myeloid and lymphoid cell types including T cells, B cells, and natural killer cells.

As used herein, the term “hematopoietic progenitor” refers to cells capable of hematopoietic transition to hematopoietic cell-types. In some embodiments, hematopoietic progenitor cells are characterized by being CD56+ cells. In some embodiments, hematopoietic progenitor cells are characterized by being CD3− cells. In some embodiments, hematopoietic progenitor cells are characterized by being CD56+/CD3− cells.

In some embodiments, one or more of the media of the culture platform is a feeder-free environment, and optionally is substantially free of cytokines and/or growth factors. In some embodiments, the cell culture media contains supplements such as serums, extracts, growth factors, hormones, cytokines and the like. Generally, the culture platform comprises one or more of stage specific feeder-free, serum-free media, each of which further comprises one or more of the followings: nutrients/extracts, growth factors, hormones, cytokines and medium additives. Suitable nutrients/extracts may include, for example, DMEM/F-12 (Dulbecco's Modified Eagle Medium/Nutrient Mixture F-12), which is a widely used basal medium for supporting the growth of many different mammalian cells; KOSR (knockout serum replacement); L-glut; NEAA (Non-Essential Amino Acids). Other medium additives may include, but not limited to, MTG, ITS, (ME, anti-oxidants (for example, ascorbic acid). In some embodiments, a culture medium of the present invention comprises one or more of the following cytokines or growth factors: epidermal growth factor (EGF), acidic fibroblast growth factor (aFGF), basic fibroblast growth factor (bFGF), leukemia inhibitory factor (LIF), hepatocyte growth factor (HGF), insulin-like growth factor 1 (IGF-1), insulin-like growth factor 2 (IGF-2), keratinocyte growth factor (KGF), nerve growth factor (NGF), platelet-derived growth factor (PDGF), transforming growth factor beta (TGF-β), bone morphogenetic protein (BMP4), vascular endothelial cell growth factor (VEGF) transferrin, various interleukins (such as IL-1 through IL-18), various colony-stimulating factors (such as granulocyte/macrophage colony-stimulating factor (GM-CSF)), various interferons (such as IFN-γ) and other cytokines having effects upon stem cells such as stem cell factor (SCF) and erythropoietin (EPO). These cytokines may be obtained commercially, for example from R&D Systems (Minneapolis, Minn.), and may be either natural or recombinant. In some other embodiments, the culture medium of the present invention comprises one or more of bone morphogenetic protein (BMP4), insulin-like growth factor-1 (IGF-1), basic fibroblast growth factor (bFGF), vascular endothelial growth factor (VEGF), hematopoietic growth factor (for example, SCF, GMCSF, GCSF, EPO, IL3, TPO, EPO), Fms-Related Tyrosine Kinase 3 Ligand (F1t3L); and one or more cytokines from Leukemia inhibitory factor (LIF), IL3, IL6, IL7, IL11, IL15. In some embodiments, the growth factors/mitogens and cytokines are stage and/or cell type specific in concentrations that are determined empirically or as guided by the established cytokine art.

### Pharmaceutical Compositions

In an aspect, the disclosure provides a pharmaceutical composition comprising the engineered immune cell of the disclosure and one or more pharmaceutically acceptable excipients or diluents.

As used herein the term “pharmaceutical composition” refers to pharmaceutically acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable diluent or excipient” or “pharmaceutically acceptable carrier” refers to an excipient, diluent, preservative, solubilizer, emulsifier, adjuvant, and/or vehicle with which an NK cell of the disclosure, is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfate; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable diluent or excipient” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

Formulations of a pharmaceutical composition suitable for administration typically generally comprise the active ingredient combined with a pharmaceutically acceptable diluents or excipients, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. Formulations may also include aqueous solutions which may contain excipients such as salts, carbohydrates and buffering agents or sterile, pyrogen-free, water. Exemplary administration forms may include solutions or suspensions in sterile aqueous solutions, for example, aqueous propylene glycol or dextrose solutions. Such dosage forms can be suitably buffered, if desired.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, and/or aromatic substances and the like which do not deleteriously interact with the formulation. In some embodiments, the pharmaceutical composition comprises said NK cells in combination with other therapeutically active agents. In some embodiments, the pharmaceutical composition comprises said NK cells in combination with antibodies specific to a disease cell phenotype. In some embodiments, the disease cell phenotype is that of a malignant cell. In some embodiments, the disease cell phenotype is that of a viral infection.

The term “combination” refers to either a fixed combination in one dosage unit form, or a kit of parts for the combined administration where one or more active compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

### Kits

In an aspect, the disclosure provides a kit comprising the engineered immune cell of the disclosure or the pharmaceutical composition of the disclosure and instructions for use.

### Method of Use

The present invention provides engineered immune cells derived from a renewable source of gene-edited iPSCs that demonstrate enhanced ADCC activity. These cells provide a promising use for standardized, off-the-shelf NK cell-based therapies in conjunction with therapeutic antibodies to effectively treat refractory malignancies and potentially other diseases, such as chronic viral infections.

In an aspect, the disclosure provides a method of treating or preventing a disease or disorder in a subject in need thereof, comprising administering the engineered immune cell of the disclosure or the pharmaceutical composition of the disclosure to the subject. In some embodiments, the disease or disorder is a malignancy. In some embodiments, the malignancy comprises a tumor-associated antigen. In some embodiments, the disease or disorder is a viral infection. In some embodiments, the viral infection comprises a viral infection-associated antigen.

The terms “subject,” “patient” and “individual” are used interchangeably herein to refer to a vertebrate, preferably a mammal, more preferably a human. Tissues, cells, and their progeny of a biological entity obtained in vivo or cultured in vitro are also encompassed. A “subject,” “patient” or “individual” as used herein, includes any animal that exhibits pain that can be treated with the vectors, compositions, and methods contemplated herein. Suitable subjects (e.g., patients) include laboratory animals (such as mouse, rat, rabbit, or guinea pig), farm animals, and domestic animals or pets (such as a cat or dog). Non-human primates and, preferably, human patients, are included.

In some embodiments, administering comprises administering a therapeutically effective amount to a subject.

As used herein, the term “amount” refers to “an amount effective” or “an effective amount” of a cell to achieve a beneficial or desired prophylactic or therapeutic result, including clinical results. As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent. In some embodiments, the engineered immune cell or pharmaceutical composition comprising said engineered immune cell of the disclosure is administered in a prophylactically effective amount.

The immune cells or pharmaceutical compositions of the disclosure may be administered in a number of ways depending upon whether local or systemic treatment is desired. The NK cells or pharmaceutical compositions are typically suitable for parenteral administration, wherein administration includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrastemal, intravenous, intranasal, intratracheal, intraarterial, intrathecal, intraventricular, intraurethral, intracranial, intratumoral, intraocular, intradermal, intrasynovial injection or infusions, intra-tumoral; and kidney dialytic infusion techniques. In some embodiments, the immune cells, or pharmaceutical compositions of the present disclosure comprise intravenous administration. In some embodiments, the immune cells, or pharmaceutical compositions of the present disclosure comprise intra-tumoral administration. In some embodiments, the immune cells, or pharmaceutical compositions are administered to a patient in a similar fashion to previous clinical work with immune cell-based therapies using unmodified peripheral blood immune, or NK, cells.

In some embodiments, the engineered immune cell or pharmaceutical composition comprising said immune cells of the disclosure are administered in combination with a combination partner. The term “combination” refers to either a fixed combination in one dosage unit form, or a kit of parts for the combined administration where the engineered immune cell, or pharmaceutical composition comprising said engineered immune cell of the disclosure, and a combination partner (e.g., another drug as explained below, also referred to as “therapeutic agent” or “co-agent”) may be administered independently at the same time or separately within time intervals. In some circumstances the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

In some embodiments, the administering further comprises administering the engineered immune cell or pharmaceutical composition comprising said engineered immune cell in combination with an antibody specific to a disease. In some embodiments, the antibody specific to a disease is an anti-CD20 antibody. In some embodiments, the anti-CD20 antibody is rituximab. In some embodiments, the antibody specific to a disease is an anti-EGFR antibody. In some embodiments, the anti-EGFR antibody is cetuximab. Such combination may lead to antibody dependent cell cytotoxicity (ADCC) and or antibody dependent cell phagocytosis (ADCP).

As used herein, “antibody” is understood to mean any antigen-binding molecule or molecular complex comprising at least one complementarity determining region (CDR) that binds specifically to, or interacts specifically with, the target antigen. The term “antibody” includes full-length immunoglobulin molecules comprising two heavy (H) chains and two light (L) chains inter-connected by disulfide bonds, as well as multimers thereof (e.g., IgM). Each heavy chain comprises a heavy chain variable region (which may be abbreviated as HCVR, VH or VH) and a heavy chain constant region. The heavy chain constant region typically comprises three domains—CH1, CH2 and CH3. Each light chain comprises a light chain variable region (which may be abbreviated as LCVR, VL, VK, VK or VL) and a light chain constant region. The light chain constant region will typically comprise one domain (CL1). The VH and VL regions can be further subdivided into regions of hypervariability, termed complementarity determining regions (CDRs), interspersed with regions that are more conserved, also referred to as framework regions (FR).

As described herein, “antigen-binding molecule” is an antibody or an antigen binding fragment thereof, as described elsewhere herein. In an embodiment, the antigen binding fragment is selected from the group consisting of a Fab fragment, scFab, Fab′, a single chain variable fragment (scFv) and a one-armed antibody.

Non-limiting examples of suitable antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated CDR such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, one-armed antibodies, diabodies, triabodies, tetrabodies, minibodies, nanobodies (e.g. monovalent nanobodies, bivalent nanobodies, etc.), and small modular immunopharmaceuticals (SMIPs), are also encompassed by the term “antigen-binding fragment,” as used herein.

As used herein, the term “complementarity determining region” (CDR) refers to the region of an immunoglobulin variable domain that recognizes and binds to the target antigen. Each variable domain may comprise up to three CDR sequences, identified as CDR1, CDR2 and CDR3.

The phrase “specifically binds” or “specific binding” refers to a binding reaction between two molecules that is at least two times the background and more typically more than 10 to 100 times background molecular associations under physiological conditions. When using one or more detectable binding agents that are proteins, specific binding is determinative of the presence of the protein, in a heterogeneous population of proteins and other biologics. Thus, under designated immunoassay conditions, the specified antigen-binding molecule binds to a particular antigenic determinant, thereby identifying its presence. Specific binding to an antigenic determinant under such conditions requires an antigen-binding molecule that is selected for its specificity to that determinant. This selection may be achieved by subtracting out antigen-binding molecules that cross-react with other molecules. A variety of immunoassay formats may be used to select antigen-binding molecules (e.g., immunoglobulins) [such that they are specifically immunoreactive with a particular antigen]. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Methods of determining binding affinity and specificity are also well known in the art (see, for example, Harlow and Lane, supra); Friefelder, “Physical Biochemistry: Applications to biochemistry and molecular biology” (W.H. Freeman and Co. 1976).

Antibodies may include, but are not limited to, monoclonal antibodies (including full length monoclonal antibodies), polyclonal antibodies, multi-specific antibodies (e.g., bi-specific antibodies), and antibody fragments so long as they exhibit the desired biological activity of binding to a target antigenic site and its isoforms of interest. The term “antibody fragments” comprise a portion of a full length antibody, generally the antigen binding or variable region thereof. The term “antibody” as used herein encompasses any antibodies derived from any species and resources, including but not limited to, human antibody, rat antibody, mouse antibody, rabbit antibody, and so on, and can be synthetically made or naturally-occurring.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The “monoclonal antibodies” may also be isolated from phage antibody libraries using the techniques known in the art.

As used herein, the term “isolated” is used to refer to molecules or cells that are removed from native environments. As used herein, the term “non-naturally occurring” is used to refer to isolated molecules or cells that possess markedly different structures than counterparts found in nature.

The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. As used herein, a “chimeric protein” or “fusion protein” comprises a first polypeptide operatively linked to a second polypeptide. Chimeric proteins may optionally comprise a third, fourth or fifth or other polypeptide operatively linked to a first or second polypeptide. Chimeric proteins may comprise two or more different polypeptides. Chimeric proteins may comprise multiple copies of the same polypeptide. Chimeric proteins may also comprise one or more mutations in one or more of the polypeptides. Methods for making chimeric proteins are well known in the art.

In some embodiments, the subject in need thereof has or is believed to have a malignancy. Many types of malignancies can develop resistance mechanisms to evade attacks from endogenous NK cells, nonlimiting examples are provided herein. In some embodiments, the malignancy may include Acute Lymphoblastic Leukemia (ALL), Acute Myeloid Leukemia (AML), Adrenocortical Carcinoma, Kaposi Sarcoma (Soft Tissue Sarcoma), AIDS-Related Lymphoma (Lymphoma), Primary CNS Lymphoma (Lymphoma), Anal Cancer, Appendix Cancer, Astrocytomas, Atypical Teratoid/Rhabdoid Tumor, Basal Cell Carcinoma of the Skin, Bile Duct Cancer, Bladder Cancer, Bone Cancer (includes Ewing Sarcoma and Osteosarcoma and Malignant Fibrous Histiocytoma), Brain Tumors, Breast Cancer, Bronchial Tumors, Burkitt Lymphoma, Carcinoid Tumor, Carcinoma, Cardiac Tumors, Atypical Teratoid/Rhabdoid Tumor, Medulloblastoma, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma, Ductal Carcinoma In Situ (DCIS), Endometrial Cancer, Ependymoma, Esophageal Cancer, Esthesioneuroblastoma, Ewing Sarcoma, Extracranial Germ Cell Tumor, Extragonadal Germ Cell Tumor, Intraocular Melanoma, Retinoblastoma, Fallopian Tube Cancer, Fibrous Histiocytoma of Bone, Osteosarcoma, Gallbladder Cancer, Gastric Cancer, Gastrointestinal Carcinoid Tumor, Gastrointestinal Stromal Tumors (GIST) (Soft Tissue Sarcoma), Germ Cell Tumors, Central Nervous System Germ Cell Tumors, Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Hepatocellular Cancer, Histiocytosis (Langerhans Cell), Hodgkin Lymphoma, Hypopharyngeal Cancer, Islet Cell Tumors, Pancreatic Neuroendocrine Tumors, Kaposi Sarcoma (Soft Tissue Sarcoma), Renal Cell Cancer, Langerhans Cell Histiocytosis, Laryngeal Cancer, Leukemia, Lip and Oral Cavity Cancer, Liver Cancer, Lung Cancer (Non-Small Cell, Small Cell, Pleuropulmonary Blastoma, and Tracheobronchial Tumor), Lymphoma, Male Breast Cancer, Malignant Fibrous Histiocytoma of Bone and Osteosarcoma, Merkel Cell Carcinoma, Mesothelioma, Metastatic Cancer, Metastatic Squamous Neck Cancer with Occult Primary, Midline Tract Carcinoma With NUT Gene Changes, Oropharyngeal Cancer, Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Chronic Myelogenous Leukemia (CML), Myeloid Leukemia, Acute (AML), Chronic Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Primary Central Nervous System (CNS) Lymphoma, Primary Peritoneal Cancer, Prostate Cancer, Recurrent Cancer, Rhabdomyosarcoma, Salivary Gland Cancer, Vascular Tumors, Small Intestine Cancer, Soft Tissue Sarcoma, T-Cell Lymphoma, Thymoma and Thymic Carcinoma, Transitional Cell Cancer of the Renal Pelvis and Ureter, Vaginal Cancer, Vulvar Cancer, or Wilms Tumor.

In some embodiments, the malignancy may comprise tumor-associated antigens. In some embodiments, the malignancy may comprise a cell marker characteristic of a malignancy. In some embodiments, the cell marker characteristic of a malignancy is a tumor-associated antigen, receptor, or other protein or structure attributed to cells with cancerous phenotypes.

Illustrative tumor-associated antigens include, but are not limited to, tumor antigens derived from or comprising any one or more of, p53, Ras, c-Myc, cytoplasmic serine/threonine kinases (e.g., A-Raf, B-Raf, and C-Raf, cyclin-dependent kinases), MAGEA1, MAGE-A2, MAGE-A3, MAGE-A4, MAGE-A6, MAGE-A10, MAGE-A12, MART-1, BAGE, DAM-6, DAM-10, GAGE-1, GAGE-2, GAGE-8, GAGE-3, GAGE-4, GAGE-5, GAGE-6, GAGE-7B, NA88-A, MART-1, MC1R, Gp100, PSA, PSM, Tyrosinase, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, 0-catenin/m, Caspase-8/m, CEA, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (e.g., such as EGFRvIII), platelet derived growth factor receptor (PDGFR), vascular endothelial growth factor receptor (VEGFR), cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notchl-4), c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin Bl, polysialic acid, MYCN, RhoC, GD3, fucosyl GM1, mesothelian, PSCA, sLe, PLAC1, GM3, BORIS, Tn, GLoboH, NY-BR-1, RGsS, SART3, STn, PAX5, OY-TES1, sperm protein 17, LCK, HMWMAA, AKAP-4, SSX2, XAGE 1, B7H3, legumain, TIE2, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, LRRN1, melanocyte melanoma lineage antigens (e.g., MART-1/Melan-A, gp75, mda-7, tyrosinase and tyrosinase-related protein), HER-2/neu, and idiotypes.

In some embodiments, the malignancy, or cells thereto, exhibit CD19, CD20, Her2, CD19, CD319/CS1, ROR1, CD20, CD5, CD7, CD22, CD70, CD30, BCMA, CD25, NKG2D ligands, MICA/MICB, carcinoembryonic antigen, alphafetoprotein, CA-125, MUC-1, epithelial tumor antigen, melanoma-associated antigen, mutated p53, mutated ras, HER2/Neu, ERBB2, folate binding protein, HIV-1 envelope glycoprotein gp120, HIV-1 envelope glycoprotein gp141, GD2, CD123, CD33, CD30, CD56, c-Met, mesothelin, GD3, HERV-K, IL-11Ralpha, kappa chain, lambda chain, CSPG4, ERBB2, WT-1, EGFRvIII, TRAIL/DR4, VEGFR2, PTK-7, B7H3, PD-L1, CD38, CLL-1, LeY, CAIX, CD133, CD171, GPC3, CEA, Ep-CAM, EphA2, FAP, HPV16-E6, IL13Ra2, MAGEA3, MAGEA4, MART1, MUC16, NY-ESO-1 and/or PSCA, CLL-1/CLEC12A, BCMA, TROP2, Nectin-4, CD79b, CD2, CD3, CD4, PD-1, KIR2DL3, ALPPL2, or CSP1.

In some embodiments, the subject in need thereof has or is believed to have a viral infection. In some embodiments, the viral infections mammalian viral infection. Examples of mammalian viral infections include, but are not limited to: infections caused by DNA Viruses (e.g., Herpes Viruses such as Herpes Simplex viruses, Epstein-Barr virus, Cytomegalovirus; Pox viruses such as Variola (small pox) virus; Hepadnaviruses (e.g, Hepatitis B virus); Papilloma viruses; Adenoviruses); RNA Viruses (e.g., HIV I, II; HTLV I, II; Poliovirus; Hepatitis A; Orthomyxoviruses (e.g., Influenza viruses); Paramyxoviruses (e.g., Measles virus); Rabies virus; Hepatitis C); Coronavirus (causes Severe Acute Respiratory Syndrome (SARS)); Rhinovirus, Respiratory Syncytial Virus, Norovirus, West Nile Virus, Yellow Fever, Rift Valley Virus, Lassa Fever Virus, Ebola Virus, and Lymphocytic Choriomeningitis Virus. In some embodiments, the viral infection is acute. In some embodiments, the viral infection is chronic.

Cells infected with a virus may present with viral infection-associated antigens. Nonlimiting examples of viral infection-associated antigens include, but are not limited to, core protein (C protein), non-structural protein 3 (NS3), non-structural protein 5 (NS5), enveloped protein (E protein), non-structural protein 4 (NS4), hemagglutinin (HA), nucleoprotein (NP), neuraminidase (NA), matrix protein 1 (M1), F protein, N protein, G protein, capsid protein (C), non-structural protein (NS), envelop protein (E), precursor membrane protein (prM), non-structural protein 1 (NS1), Gag, Env, Tat, Pol, Nef, Vif, capsid protein P1 (VP2), capsid protein P1 (VP1), and capsid protein P1 (VP3).

### Cellular Culture

In an aspect, the disclosure provides a cellular culture comprising genetically modified ADAM17-deficient Natural Killer (NK) cells. In embodiments, the NK cells have been produced from induced pluripotent stem cells (iPSCs). In embodiments, the NK cells have been produced from peripheral blood cells or cord blood cells. In embodiments, the NK cells are human NK cells.

In some embodiments, the NK cells exhibit enhanced antibody-dependent cellular cytotoxicity (ADCC) as compared to non-genetically modified NK cells.

In embodiments, the NK cells express cell surface markers CD56, NKG2D, NKp44, and NKp46. In embodiments, the invention provides that the NK cells express increased cell surface markers CD16, TNF-α, and CD62L as compared to non-genetically modified NK cells.

In embodiments, the NK cells have been genetically modified to be ADAM17-deficient with CRISPR, TALEN, ZFN or other gene editing techniques.

In an aspect, the disclosure provides a pharmaceutical composition comprising NK cells from the culture of genetically modified ADAM17-deficient NK cells as described herein.

In an aspect, the disclosure provides a method of treating a subject in need comprising administering to the subject an effective amount of a pharmaceutical composition as described herein. In embodiments, the invention provides that the subject in need has a NK-resistant cancer. In embodiments, the invention provides that the subject in need has a chronic viral infection.

In embodiments, the administration further includes antibodies specific for a diseased cell. In embodiments, the invention provides that the administration further includes antibodies specific for CD20.

In an aspect, the disclosure provides a method of manufacturing a NK cell culture as described herein comprising genetically modifying a cell to be ADAM17-deficient. In some embodiments, the cell is an induced pluripotent stem cell (iPSC).

The disclosure is now described with reference to the following Examples. These Examples are provided for the purpose of illustration only and the disclosure should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the methods of the present disclosure and practice the claimed methods. The following working examples therefore, specifically point out embodiments of the present disclosure, and are not to be construed as limiting in any way the remainder of the disclosure.

### EXAMPLES

The materials and methods employed in these experiments are now described.

### Example 1: Production of ADAM17-Deficient iPSC-Derived NK Cells

The present invention is the first to describe the deletion of ADAM17 expression in human iPSCs to generate NK cells with enhanced antibody-dependent cell mediated cytotoxicity (ADCC). Additionally, this approach leads to not only improved CD16 expression, but also increased expression of TNF-α, CD62L and potentially other cell surface molecules that are subject to ADAM17-mediated cleavage.

Previous studies have demonstrated that improved CD16 expression in iPSC-derived NK cells via production of a cleavage resistant CD16 molecule does lead to improved ADCC activity, and this approach has now entered clinical trials (Fate Therapeutics FT516 and FT596 clinical products). However, this approach only leads to increased CD16 expression and not other ADAM17-mediated cleavage proteins.

Initial study that identified CD16 cleavage by ADAM17 demonstrated that inhibiting ADAM17 activity led to increase in cytokine production by NK cells when co-incubated with antibody-coated target cells, see Romee, et al. (2013) Blood. 121(18):3599-608. To block ADAM17 activity, a small molecule inhibitor (BMS566394) can be used, see Mishra et al. (2021) Frontiers in Immunology 12(711621):1-9. This study was performed on peripheral blood NK cells. As ADAM17 is expressed on a number of different cell types, use of a small molecule inhibitor in a clinical setting to block ADAM17 activity will result in non-NK-specific effects that may negatively affect therapeutic efficacy. The genetic knockout approach of the present invention uses iPSC-NK and specifically affects ADAM17 on the NK cells without affecting physiological ADAM17 activity in other cells.

A separate study reported the use of CRISPR/Cas9 targeting ADAM17 along with the delivery of a homologous recombination template using adeno-associated virus to interrupt ADAM17 expression in peripheral blood NK cells, see Pomeroy et al. (2020) Molecular Therapy 26(1):52-63. They report that ADAM17 disrupted NK cells display improved cytokine product and cytotoxicity against cancer cells. The strategy reported in this article also uses peripheral blood NK cells, which is prone to donor variability and generates a heterogeneous gene-modified NK cell population. The ADAM17-deficient iPSC-NK from the present invention provides a more stable, uniform platform to generate consistent gene-edited NK cells with enhanced anti-tumor activity.

As a strategy to enhance NK cell ADCC activity, ADAM17-deficient (ADAM17-KO) iPSC-derived NK cells were generated. Briefly, ADAM17-KO iPSCs were generated using clustered regularly interspaced short palindromic repeats/Cas9 (CRISPR/Cas9) with a short guide RNA (sgRNA) targeting Exon 1 of ADAM17 on iPSCs derived from umbilical cord blood cells. After transfection of CRISPR/Cas9 with ADAM17-targeting sgRNA, single cells clones were isolated and assessed for mutations in ADAM17 by PCR and Sanger sequencing. Transfected cells not containing the mutation were used as a wildtype control. One clone containing homozygous mutation in ADAM17 was expanded and verified for lack of surface ADAM17 protein expression using flow cytometry compared to wildtype.

ADAM17-KO iPSCs successfully differentiate into hematopoietic progenitor cells, then to NK cells that uniformly express typical NK cell surface markers including CD56, CD94, NKG2D, NKp44, and NKp46. ADAM17-KO iPSC-NKs are functional and kill K562 erythroleukemia cells comparably to WT iPSC-NK and healthy donor-derived PB-NK cells in vitro. Surprisingly, upon differentiation, ADAM17-KO iPSC-NK cells express −20% lower CD16a surface expression compared to WT iPSC-NK cells. We therefore used magnetic cell separation to positively select for CD16a + to enrich for ADAM17-KO cells capable of ADCC. After enrichment, ADAM17-KO cells stably expressed CD16a+ cells over 6 weeks of expansion in culture using artificial antigen presenting cells. WT iPSC-NKs and PB-NKs rapidly lose CD16a surface expression upon stimulation with phorbol esters, while ADAM17 KO iPSC-NK cells maintain over 90% CD16a expression after this stimulation. Additionally, a significantly higher proportion of ADAM17-KO iPSCs express TNF-α and CD62L (L-Selectin) two other known ADAM17 substrates, on the cell surface after stimulation with phorbol esters for 4 hours compared to WT iPSC-NK and PB-NK.

ADCC activity was assessed by observing CD107a and IFNγ expression upon incubation with tumors typically resistant to NK cell killing, and therapeutic antibodies that bind to antigens expressed on these tumor cells. When co-incubated with RAJI B-lymphoma cells, CD16a+ADAM17-KO iPSC-NK cells mediate increased CD107a and IFNγ expression in the presence of the anti-CD20 antibody rituximab, compared to CD16a+WT iPSC-NK and PB-NK cells. Similarly, CD16a+ADAM17-KO iPSC-NK cells upregulate increased CD107a and IFNγ expression when co-incubated with CAL27 squamous cell carcinoma cells in the presence of the anti-EGFR antibody Cetuximab, compared to CD16a+WT iPSC-NK and PB-NK. Long-term (24 hour) cytotoxicity assay against RAJI and Ca127 cells in the presence of rituximab and cetuximab, respectively, demonstrates higher cytotoxicity in CD16a+ADAM17-KO iPSC-NK cells compared to CD16a+WT iPSC-NK and CD16a+PB-NK cells over time.

### REFERENCES

1. Zhu H, Blum R, Bjordahl R, Gaidarova S, Rogers P, Lee T T, Abujarour R, Bonello G B, Wu J, Tsai P F, Miller J S, Walcheck B, Valamehr B, Kaufman D S. (2020). Pluripotent stem cell-derived N K cells with high-affinity non-cleavable CD16a mediate improved anti-tumor activity. Blood. 135(6):399-410.
2. Jing Y, Ni Z, Wu J, Higgins L, Markowski T W, Kaufman D S, Walcheck B. (2015). Identification of an ADAM17 Cleavage Region in Human CD16 (FcγRIII) and the Engineering of a Non-Cleavable Version of the Receptor in N K Cells. PLoS One.
3. Romee R, Foley B, Lenvik T, Wang Y, Zhang B, Ankarlo D, et al. N K cell CD16 surface expression and function is regulated by a disintegrin and metalloprotease-17 (ADAM17). Blood. 2013; 121(18):3599-608.
4. Pomeroy E J, Hunzeker J T, Kluesner M G, Lahr W S, Smeester B A, Crosby M R, et al. A Genetically Engineered Primary Human Natural Killer Cell Platform for Cancer Immunotherapy. Mol Ther. 2020; 28(1):52-63.

1. An engineered immune cell, comprising an inactivating mutation in an endogenous A-Disintegrin-And-Metalloproteinase 17 (ADAM17) gene.

2. The engineered immune cell of claim 1, wherein the engineered immune cell is an induced pluripotent stem cell (iPSC)-derived immune cell.

3. The engineered immune cell of claim 1, wherein the engineered immune cell is a peripheral blood (PB)-derived immune cell.

4. The engineered immune cell of claim 1, wherein the engineered immune cell is a cord blood (CB)-derived immune cell.

5. The engineered immune cell of claim 1, wherein the engineered immune cell is a natural killer (NK) cell.

6. The engineered immune cell of claim 1, wherein the ADAM17 inactivating mutation is a knockout of an endogenous ADAM17 gene.

7. The engineered immune cell of claim 6, wherein the engineered immune cell is a ADAM17^{-/-} immune cell.

8. The engineered immune cell of claim 1, wherein the ADAM17 inactivating mutation is a knockdown of an endogenous ADAM17 gene.

9. The engineered immune cell of claim 1, wherein the engineered immune cell is CD56+, CD94+, NKG2D+, NKp44+, and NKp46+.

10. The engineered immune cell of claim 1, wherein the engineered immune cell has increased expression of CD16a, TNF-α, and CD62L (L-selectin) as compared to a non-engineered immune cell after stimulation with a stimulating agent.

11. The engineered immune cell of claim 1, wherein expression of CD16a in the engineered immune cell is stably maintained for a period of at least about 6 weeks during expansion in a culture.

12. The engineered immune cell of claim 1, wherein the immune cell is a human immune cell.

13. A purified cell composition comprising one or more of the engineered immune cell of claim 1.

14. The purified cell composition of claim 13, wherein at least about 71% of engineered immune cells express tumor necrosis factor-alpha (TNF-α) after stimulation with a stimulating agent.

15. The purified cell composition of claim 13, wherein at least about 42% of engineered immune cells express CD62L (L-selectin) after stimulation with a stimulating agent.

16. The purified cell composition of claim 13, wherein the engineered immune cells have increased expression of CD107a and interferon gamma (IFNγ) as compared to non-engineered immune cells after co-incubation with a disease cell line and an antibody specific to said disease cell line.

17. The purified cell composition of claim 16, wherein the disease cell line is a B-lymphoma cell line and the antibody specific to said disease cell line is an anti-CD20 antibody.

18. The purified cell composition of claim 17, wherein the anti-CD20 antibody is rituximab.

19. The purified cell composition of claim 17, wherein the engineered immune cells exhibit at least about 45% increased expression of CD107a.

20. The purified cell composition of claim 17, wherein the engineered immune cells exhibit at least about 36% increased expression of IFNγ.

21. The purified cell composition of claim 16, wherein the disease cell line is a squamous cell carcinoma cell line and the antibody specific to said disease cell line is an anti-EGFR antibody.

22. The purified cell composition of claim 21, wherein the anti-EGFR antibody is cetuximab.

23. The purified cell composition of claim 21, wherein the engineered immune cells exhibit at least about 29% increased expression of CD107a.

24. The purified cell composition of claim 21, wherein the engineered immune cells exhibit at least about 39% increased expression of IFNγ.

25. The purified cell composition of claim 13, wherein the engineered immune cells exhibit enhanced antibody-dependent cellular cytotoxicity (ADCC) as compared to non-engineered immune cells.

26. A method of making the engineered immune cell of claim 1 comprising:
a) introducing an inactivating mutation in an endogenous A-Disintegrin-And Metalloproteinase 17 (ADAM17) gene into a stem cell; and
b) differentiating the stem cell into an immune cell.

27. The method of claim 26, wherein the stem cell is an induced pluripotent stem cell (iPSC), peripheral blood cell, or a cord blood cell.

28. The method of claim 26, wherein the immune cell is a natural killer cell (NK) cell.

29. A pharmaceutical composition comprising the engineered immune cell of claim 1 and one or more pharmaceutically acceptable excipients or diluents.

30. A kit comprising the engineered immune cell of claim 1 and instructions for use.

31. A method of treating or preventing a disease or disorder in a subject in need thereof, comprising administering the engineered immune cell of claim 1 to the subject.

32. The method of claim 31, wherein the disease or disorder is a malignancy.

33. The method of claim 32, wherein the malignancy comprises a tumor-associated antigen.

34. The method of claim 31, wherein the disease or disorder is a viral infection.

35. The method of claim 34, wherein the viral infection comprises a viral infection-associated antigen.

36. The method of claim 31, wherein the administering further comprises administering the engineered immune cell or pharmaceutical composition comprising said engineered immune cell in combination with an antibody specific to a disease.

37. The method of claim 36, wherein the antibody specific to a disease is an anti-CD20 antibody.

38. The method of claim 37, wherein the anti-CD20 antibody is rituximab.

39. The method of claim 36, wherein the antibody specific to a disease is an anti-EGFR antibody.

40. The method of claim 39, wherein the anti-EGFR antibody is cetuximab.

41. A cellular culture comprising genetically modified ADAM17-deficient Natural Killer (NK) cells.

42. The cellular culture of claim 41, wherein the NK cells have been produced from induced pluripotent stem cells (iPSCs).

43. The cellular culture of claim 41, wherein the NK cells have been produced from peripheral blood cells or cord blood cells.

44. The cellular culture of claim 41, wherein the NK cells are human NK cells.

45. The cellular culture of claim 41, wherein the NK cells exhibit enhanced antibody-dependent cellular cytotoxicity (ADCC) as compared to non-genetically modified NK cells.

46. The cellular culture of claim 41, wherein the NK cells express cell surface markers CD56, NKG2D, NKp44, and NKp46.

47. The cellular culture of claim 41, wherein the NK cells express increased cell surface markers CD16, TNF-α, and CD62L as compared to non-genetically modified NK cells.

48. The cellular culture of claim 41, wherein the NK cells have been genetically modified to be ADAM17-deficient with CRISPR, TALEN, or ZFN.

49. A pharmaceutical composition comprising NK cells from the culture of genetically modified ADAM17-deficient NK cells of claim 41.

50. A method of treating a subject in need comprising administering to the subject an effective amount of a pharmaceutical composition of claim 49.

51. The method of claim 50, wherein the subject in need has a NK-resistant resistant cancer.

52. The method of claim 50, wherein the subject in need has a chronic viral infection.

53. The method of claim 50, wherein the administration further includes antibodies specific for a diseased cell.

54. The method of claim 50, wherein the administration further includes antibodies specific for CD20.

55. A method of manufacturing a NK cell culture of claim 41 comprising genetically modifying a cell to be ADAM17-deficient.

56. The method of claim 55, wherein the cell is an iPSC.
